(19) United States
(12) Patent Application Publication
(10) Pub. No.: US 2005/0265953 A1
(43) Pub. Date: Dec. 1, 2005

(54) Method of obtaining phytoalexins

(76) Inventors: Rachid Ennamany, Villenave D'Ornon (FR); Jean-Michel Merillon, Martillac (FR)

Correspondence Address: HOVEY WILLIAMS LLP, 2405 GRAND BLVD., SUITE 400, KANSAS CITY, MO, 64108, US

(21) Appl. No.: 10/943,698

(22) Filed: Sep. 17, 2004

Related U.S. Application Data
(63) Continuation-in-part of application No. PCT/IB03/01020, filed on Mar. 20, 2003.
(63) Continuation-in-part of application No. PCT/IB02/03971, filed on Sep. 26, 2002.

(30) Foreign Application Priority Data
Mar. 20, 2002 (FR) 02/03423

(51) Int. Cl. A61K 7/06; A61K 35/78

(52) U.S. Cl. 424/74; 424/725

(57) ABSTRACT

Topical composition containing at least one comminuted product of elicited dedifferentiated plant cells, whereby said dedifferentiated plant cells are elicited in vitro in a culture in order to synthesise at least one phytoalexin, and whereby the communited product of elicited dedifferentiated plant cells are dispersed in said composition.

This application is a Continuation in Part of PCT/IB03/01020 filed on Mar. 20, 2003 (published in French under number WO03/077881 on Sep. 25, 2003) hereby incorporated by reference, claiming the benefit of the priority of French patent application FR 02/03423 filed on Mar. 20, 2002 hereby incorporated by reference, as well as of PCT/IB02/03971 filed on Sep. 26, 2002 (published in French under number WO03/077880 filed on Sep. 25, 2003) hereby incorporated by reference.

### BRIEF DESCRIPTION OF THE INVENTION

This invention relates to a composition for topical use, particularly a cosmetic composition, which is rich in metabolites produced by dedifferentiated plant cells. The invention relates in particular to a composition containing dedifferentiated plant cells which are elicited and which are then partially or completely dried, preferably freeze-dried, and are comminuted and dispersed in said composition.

The expression “dedifferentiated plant cells” should be understood to mean any plant cell which exhibits none of the features of a particular specialised cell classification, and which is capable of living by itself and not in dependence on other cells.

Dedifferentiated plant cells can be obtained from plant material which is derived from a whole plant or from part of a plant, such as leaves, stems, flowers, petals, roots, fruit, skin, the envelope protecting them, seeds, anthers, sap, thorns, buds, peel, berries and mixtures thereof.

Dedifferentiated plant cells are preferably obtained from peel, leaves, buds and from the skin of fruit, particularly from fruit cuticles.

Dedifferentiated plant cells which can be used according to the invention can be obtained from plants obtained by in vivo culture or derived from in vivo culture.

The expression “in vivo culture” should be understood to mean any classical type of culture, i.e. in soil, in the fresh air, in a greenhouse or in a soil-free or hydroponic environment.

The expression “in vitro culture” should be understood to mean the all the techniques known to one skilled in the art which enable a plant or a part of a plant to be obtained artificially. The pressure of selection imposed by the physicochemical conditions during the growth of plant cells in vitro enables a standardised plant material to be obtained which is free from contaminants and is available all year round, in contrast to plants cultivated in vivo.

According to the invention, dedifferentiated plant cells are preferably used which are derived from culture in vitro.

The dedifferentiated plant cells which can be used according to the invention can be obtained by any method which is known from the prior art. Methods which can be cited in this respect include those described by E. F. George and P. D. Sherrington in Plantation Propagation by Tissue Culture, Handbook and Directory of Commercial Laboratories (Exegetics Ltd. 1984).

The culture media which can be used according to the invention are those which are generally known to one skilled in the art. Examples which can be cited include the media of Gamborg, Murashige and Skoogs, Heller, White etc. Complete descriptions of these media are given in “Plantation Culture Media: Formulations and Uses” by E. F. George, D. J. M. Puttock and H. J. George (Exegetics Ltd. 1987, Volumes 1 & 2).

According to the invention, the cultivated, dedifferentiated plant cells are preferably prepared on the medium of Murashige and Skoog.

### PRIOR ART

FR 2795637 discloses a cosmetic composition containing an extract of dedifferentiated plant cells to avoid odour problems. This composition contains an extract of plant cells which are dedifferentiated but not elicited, so that this composition has a low content of secondary metabolites or phytoalexins, is or even substantially free from such compounds. Moreover, this document describes the use of aqueous extracts obtained after comminution of the cells in their culture medium followed by the removal of the particles in suspension, with an unavoidable loss of metabolites bound to the particles in suspension. In order to remove proteases, and oxidases in particular, this document also recommends the use of filters which retain molecules with a molecular weight higher than 100,000 daltons, which thus results in the loss from the final extract of all metabolites with a molecular weight higher than this weight, which can prove to be of great interest to the cosmetics industry. Furthermore, in order to eliminate problems due to oxidation this document recommends the addition of stabilisers, particularly cysteine, and/or of sulphur-containing derivatives, which inevitably results in the purity of the extract being reduced during subsequent filtration stages. The methods described in this document necessitate the use of complicated means for obtaining extracts, the purity (numerous additives) and the quality and concentration (of metabolites) of which are not optimal. Moreover, the numerous stages necessary to obtain extracts by this method result in increased costs and in the risk of contamination due to the numerous manipulations and additives employed.

Cultures of dedifferentiated cells are known, as are the mechanisms of elicitation of these cells followed by extraction stages and by various filtrations followed by freeze-drying in order to incorporate the extracts obtained in a cosmetic or pharmaceutical preparation. Such methods are described, for example, in U.S. Pat. No. 4,241,536; EP 378 921, WO 88/00968, EP 1 203 811, etc. for species of various plants. The content of these documents is incorporated in the present description by reference in order to describe culture media, plant species, possible elicitors, etc.

Currently, despite the expertise and know-how of industries in the field of plant extraction, and despite the progress of organic chemistry, several extraction stages are necessary in order to obtain a plant raw material.

Several disadvantages are associated with these extraction stages:
loss of the tertiary structure of the isolated molecules,
the presence of various solvents in the final product,
substrate heterogeneity, which necessitates refined extractions using increasingly toxic solvents,
the quality of the extract depends on the physiological state of the plant when harvested,
production of the extract is limited by the seasons.

Due to these limiting factors and to the renewal of consumer interest in everything which is of natural origin, several attempts to obtain cells have been made. To date, two main methods have been employed:
The culture of cells from unicellular organisms or microorganisms, which is a not very original technique based on the reproduction of the conditions of normal life. However, these organisms are primitive and do not develop any secondary metabolism, which is the source of the active constituents of most interest.
Obtaining cells from fruit (fresh cells) after enzymatic digestion. The limitations of this method are that the fruit is not aseptic and can contain residues of pesticides (fungicides, herbicides, insecticides, etc.). Moreover, the enzymes (cellulases, pectinases, etc.) which are used in significant amounts (2%, w/w) for the digestion of plant walls and for obtaining cells without a wall (protoplasts) are found in the final product. The enzymes used can also alter the quality of the metabolites. Finally, the use of this technique only enables protoplasts (cells without a cell wall) to be recovered; these are fragile structures which are not capable of orienting their metabolism.
   The Invention

The inventors have developed an innovative, controlled technology which ensures the quality and authenticity of the products. It involves placing cells of dedifferentiated higher plants in a culture.

In fact, and for the first time, an industrial process is proposed which enables cells to be obtained from higher plants by a method which avoids any modification of their genetic heritage, allowing the cell to retain its physiological features.

Maintenance of the various strains is ensured by regular subculturing, with total control of the different conditions of culture.

The importance of this method is that it enables the culture of dedifferentiated plant cells to be effected on a large scale whilst responding to the needs of the industry, in particular:
Preservation of the tertiary structure of the molecules,
Absence of solvent and residues,
Substrate homogeneity,
Continuous processing, regardless of the cycle of the seasons,
Retention of biological and physiological characteristics without the addition of preservatives,
Complete absence of pollutants,
Standardised, reproducible production with regard to metabolite quality and concentration,
The use of these plant suspensions after direct freeze-drying at a temperature less than 30° C. This technique enable a very fine powder to be obtained which is suitable for dispersion in cosmetic compositions (creams, ointments, lotions, etc.). These cells are capable of directly releasing the active constituents which they contain without passage through an extraction stage using organic solvents (elimination of the risk of residues). However, the product of freeze-drying is preferably subjected to comminution to prevent any agglomeration of particles.
The use of the cell extracts solely after ultrasonic treatment and centrifugation.

This technology provides a useful, innovative alternative to conventional solvent extraction methods. The possibility of naturally orienting (by elicitation) the synthesis of metabolites without undermining the genetic integrity of the cells constitutes a guarantee of quality and authenticity.

Quite surprisingly, the inventors have discovered that after elicitation, drying and comminution the cells can be directly incorporated or dispersed in a cosmetic and/or pharmaceutical composition. The composition according to the invention contains cell membranes, cytoplasmic organisms and vacuole material. In particular, this method has the advantage of deactivating oxidising enzymes without additions of additives or chemical products. Another aspect of the invention enables the production of phytoalexins to be concentrated and directed without quantitative or qualitative losses due to extraction and filtering stages. A particular aspect of the invention is that it avoids extraction and filtering stages and enables a comminuted cell material to be obtained which is devoid of additives, solvents and residues, said comminuted product being capable of being dispersed directly in a cosmetic composition. The composition according to the invention contains at least one comminuted, dedifferentiated plant cell material which is elicited in a culture in vitro in order to synthesise at least one phytoalexin, said elicitation in order to synthesise at least one phytoalexin advantageously being effected after a stage of culture in vitro of plant cells without elicitation, wherein said comminuted product containing at least one phytoalexin comprises at least 95%, advantageously at least 97%, preferably at least 99% by weight of the entirety of the dry materials derived from the comminuted plant cells which are dedifferentiated and elicited in vitro, said comminuted product being dispersed in said composition or being in a form suitable for being dispersed in said composition. In particular, the comminuted product contains substantially all the dry materials (after extraction of the water present in the cells) of the dedifferentiated, elicited, comminuted plant cells. However, a comminuted product such as this, which is rich in phytoalexin in relation to the content of cells which are not elicited, contains all the natural materials which are present in the cells.

Amongst its other aims, the object of the present invention is to propose a simple method which excludes the use of additives and chemical products whilst retaining the natural character of the cells obtained. Moreover, elicitation by physical means enables the production of metabolites which are sought by the cosmetics industry to be directed and concentrated. The particular aspect of the invention comprising retaining the integrity of the cells obtained and eliciting them firstly enables the concentration and the quality of the metabolites obtained to be optimised and secondly enables oxidation problems to be solved by deactivating the enzymes by a simple drying operation (freeze-drying) without the addition of additives and without losses due to filtration, and finally enables the comminuted product obtained to be dispersed directly in a cosmetic preparation.

The expression “elicitation in the culture medium” should be understood to mean subjecting the cells to stress or attack (biological, chemical or physical) in their culture medium in order to trigger one or more defence mechanisms.

During the whole of their development, plants are subjected to continuous attacks by their environment. However, they often appear capable of resisting these external attacks naturally, by virtue of the presence or activation of defence mechanisms (Hammond-Kosack and Jones, 1996). Thus, although some of these mechanisms are inherent and provide a physical and chemical barrier to attack, others are only induced after an attack by a harmful agent.

As soon as a plant detects a pathogen, it employs one of the most efficient natural defence systems: the response of hypersensitivity. At the site of penetration of the pathogen, this reaction, which is rapid and violent, results in the death of the first infected cells and in the appearance of a small necrotic zone, which thus isolates the attacked cells from the rest of the plant (Dangi et al., 1996; Lamb and Dixon, 1997). The triggering of this response depends on the specific recognition of the pathogen agent by the host plant. In fact, the attacked plant recognises, via a (receptor) protein, a protein produced by the pathogen and termed an elicitor. In the plant, the genes which code for receptor proteins are called resistance genes (R genes), and in the pathogen the genes which code for the elicitor molecules are called avirulence genes (Avr gene): what is involved here is a gene-for-gene relationship or an R-Avr relationship (Hammond-Kosack, 1996). Other molecules, which are classified as general elicitors, are also capable of initiating (in a less specific manner than the elicitors cited previously) this defence reaction of the host plant. These are most often oligosaccharides released by the pathogen (exogenous elicitors) or the plant cell (endogenous elicitors), (Scheel and Parker, 1996).

This hypersensitivity reaction is followed by the activation of an intense defence reaction in the cells adjacent to the infected zone; this is the local reaction. Finally, there is the emission of various warning signals to all the other organs of the plant, which enable it to react more rapidly and more efficiently to a new attack; this is termed a systemic reaction.

During these reactions, three categories of defence systems can be activated:
the formation of a healing epidermis and reinforcement of the walls (lignification, etc.) (Dai et al., 1995);
the synthesis of defence proteins or “Pathogenesis Related” (PR) proteins discovered in 1970 by the tobacco industry. These PRs, for example, include protease inhibitors (Ryan, 1992), hydrolytic enzymes such as chitinases or β-1,3-glucanases (Derckel et al., 1996; Robinson et al., 1997, Kraevas et al., 1998; Salzman et al., 1998; Renault et al., 2000);
and the synthesis of secondary metabolites of the phytoalexin type. Of these secondary metabolites, more than 300 phytoalexins have already been characterised. They form part of a large spectrum of different chemical classes which include coumarins, benzofurans, terpenes, alkaloids, certain polyphenols (Smith, 1996), etc.

The implementation of defence reactions of plants involves a whole panoply of transduction signals which result in the rapid induction of the expression of defence genes. Thus, the recognition of the pathogen by the host plant activates a cascade of signals in the attacked cells, such as the phosphorylation of proteins by protein kinases, the flow of ionic species (Ca²⁺), the formation of reactive oxygenated species (Coté and Hahn, 1994; Shibuya et al., 1996; Benhamou, 1996), etc.

Moreover, the attacked cells are capable of producing alarm signals which are transmitted to adjacent cells (local reaction) as well as to the whole plant, and which thus generate the systemic reaction phenomenon, as stated in the previous paragraph.

The most studied mechanism of systemic resistance is the phenomenon of SAR or “systemic acquired resistance.” The term SAR was defined by Ross in 1961. It describes the appearance of the resistance of a plant following an attack by a pathogen, both in the infected parts and in the healthy parts of the plant. In general, it is developed after the appearance of necrotic lesions around the inoculation site. This localised hypersensitivity response restricts the pathogen to within and around the site of infection, and appears to make the plant more resistant to attack by various organisms (Ryals et al., 1996). How are plant parts remote from the infection site capable of acquiring this resistance? In 1966 Ross developed the idea of the existence of signal molecules which, at low concentrations, are capable of activating defence mechanisms in tissues remote from the infected zone.

Three types of molecules can act as intra- and intercellular alarm signals in plants over short or long distances: salicylic acid, ethylene and jasmonates.

The most studied and best known transduction mechanism in the development of ASR is that which involves salicylic acid (SA) (Delaney et al., 1994; Ryals et al., 1996).

On the one hand, the acquisition of resistance of some plants is directly associated with an increase in the endogenous synthesis of SA or derivatives thereof (Malamy et al., 1990; Metraux et al., 1990; Rasmussen et al., 1991; Smith-Becker et al., 1998). In this type of response, when synthesis of SA is initiated, transduction of the signal takes place long-distance, either by direct transport of SA (Shulaev et al., 1995) or by the conversion of the latter into methyl salicylate (MeSa), which is a volatile signal molecule capable of initiating the appearance of resistance in the healthy tissues of the infected plant and also in adjacent plants (Shulaev et al., 1997). Moreover, evidence of the role played by SA in triggering ASR is linked with the use of transgenic tobacco plants which express the bacterial gene NahG. This gene, which codes for salicylate hydroxylase, deactivates SA by converting it into catechol, thus rendering transformed plants incapable of developing an ASR (Gaffney et al., 1993).

On the other hand, certain studies have shown that the application of exogenous SA is capable of inducing resistance to different lesions caused by a bacterium, a virus or a pathogen. Thus, in tobacco, a reduction is observed of symptoms associated with the inoculation of tobacco mosaic virus after treatment of the plants by SA (White et al., 1983). In the same way, SA is capable of stimulating the biosynthesis of different PR proteins which are normally produced in the event of an ASR (Renault et al., 1996; Narusaka et al., 1999).

Various studies have shown that some defence reactions can be activated regardless of the presence of SA. Thus, other types of molecules were recognised as ASR signal molecules (Enyedi et al., 1992; Pieterse et al., 1999). These reactions mainly involve the intervention of two plant growth regulators, namely ethylene and jasmonic acid (JA), as systemic defence induction signals. In fact, these two types of molecules can be produced rapidly and endogenously when a plant is subjected to an attack, and thus result in the appearance of a resistance. Moreover, they are also capable of inducing the expression of certain PRs and/or the production of phytoalexins.

Ethylene is a volatile phytohormone which intervenes in many physiological processes of plants. Its role in plant defence phenomena has been demonstrated by several teams. Studies have shown that the attack of a plant by a pathogen or a herbivore can be correlated with the induction or increase of endogenous ethylene synthesis in the host plant (O'Donnell et al., 1996; Popp et al., 1997; Lund et al., 1998). Moreover, just like SA, the application of exogenous ethylene is also capable of stimulating the synthesis of PR proteins (Ward et al., 1991; Penninckx et al., 1996; Yang et al., 1997). Finally, the activation of defence proteins in Arabidopsis in response to a pathogen can be blocked in the case of mutants which are deficient in the perception of this signal (Pennickx et al., 1998).

Jasmonic acid and its methyl ester, methyl jasmonate (MeJa), are natural compounds of the cyclopentanone type analogous to animal prostaglandins with regard to their biosynthesis and their function (Creelman et al., 1992; Mason et al., 1993; Sadka et al., 1994). They are derived from fatty acids and are synthesised by the lypoxygenase-dependent oxidation of α-linolenic acid. This is termed the octadecanoic route.

Apart from their role in various physiological functions (Stawick, 1992), these compounds have recently been identified as intracellular signalling molecules which are synthesised in response to biotic or abiotic stress and which result in defence responses in plants, such as the biosynthesis of phytoalexins or defence proteins (Gundlach et al., 1992; Blechert et al., 1995; Creelman et al., 1995; Doares et al., 1995; Conconi et al., 1996a and 1996b; Ignatov et al., 1996; Baldwin et al., 1997).

On the one hand, the application of MeJa, which is a volatile derivative of jasmonic acid, induces the production of numerous secondary metabolites in vivo or in vitro (in cell cultures), some of which play the role of defence compounds. In fact, this ester is capable of eliciting the biosynthesis of furanocoumarins in celery leaves (Miksch and Boland, 1996), of momilactone A in cell cultures of rice (Nojiri et al., 1996), of alkaloids of young plants or cell cultures of Catharanthus roseus (Aerts et al., 1996; Gantet et al., 1997) and of young plants of Cinchona ledgeriana (Aerts et al., 1994; 1996). Other precursor derivatives of JA, such as 12-oxo-phytodienoic acid, are capable of acting on the biosynthesis of numerous secondary metabolites.

The inductive effect of these compounds is often preceded by activation of the expression of genes or the synthesis of enzymes which intervene in the biosynthesis of these various metabolites. These enzymes include phenylalanine ammonialyase (PAL), 4-coumarate-CoA ligase (4CL), chalcone synthase (CHS), dihydroflavonol-4-reductase (DFR), polyphenol oxidase (PPo), bercaptol methyl transferase (BMT), tyrosine/dopa decarboxylase (TYDC), etc. (Dittrich et al., 1992; Gundlach et al., 1992; Mizukami et al., 1993; Tamari et al., 1995; Ellard-Ivey et al., 1996; Facchini et al., 1996; Ignatov et al., 1996; Lee et al., 1997; Yasaki et al., 1997; Constanbel and Ryan, 1998).

On the other hand, exposure of plants to MeJa vapours enables defence mechanisms to be elicited which are similar to those induced by insects, herbivores, a wound or UV (Farmer and Ryan, 1990; 1992; Conconi et al., 1996b; Ozawa et al., 2000). Thus jasmonates, particularly MeJa, are capable of inducing the biosynthesis of defence and stress proteins, which are termed JIPs (Jasmonate Induced Proteins), (Reinbothe et al., 1994).

Moreover, the treatment of tomato, potato or alfalfa plants by MeJa, induces the expression of protease inhibitors (Farmer and Ryan, 1990, 1992; Hildmann et al., 1992; Pena-Cortes et al., 1992; Lee et al., 1996), the biosynthesis of thionine in barley (Reinbothe et al., 1997), of proteins rich in proline or Vsp (vegetative storage proteins) of soya beans (Stawick et al., 1991; Creelman et al., 1992; Mason et al., 1992, 1993; Berger et al., 1995).

Finally, it is also involved in the regulation of the biosynthesis of proteins which participate in the transduction of signals in response to a stress, such as lypoxygenases, for example (Saravitz and Siedow, 1996) or systemine (Reinbothe et al., 1994; Bergey et al., 1996).

Therefore, it is not surprising that various teams have shown a reduction in the incidence of some diseases following the treatment of the plant concerned by MeJa vapours (Cohen et al., 1993; Meir et al., 1998; Thomma et al., 1998; Vijayan et al., 1998; Thomma et al., 2000). Moreover, J. S. Thaler (1999) has shown that, for numerous plants, various defence mechanisms against attack by herbivores are induced via the octadecanoic route and are thus involved in the attraction of natural enemies. For example, this demonstrates that the treatment of plants by JA increases the parasitism of pest caterpillars.

In the vine, the signalling mechanisms involved in the expression of defence reactions are still not well known. However, the synthesis occurs of three types of defence molecules (lignin, defence proteins and phytoalexins). In particular, the role of phytoalexins is played by a family of original compounds, namely polyphenols (Deloire et al., 2000).

Present in more or less large amounts in all the organs of the plant, phytoalexins can be induced in leaves and berries. This type of induction is designated by the term “elicitation”. Elicitation factors (or elicitors) can have different origins. Elicitation can take the form of:
biotic elicitation, for example on an attack by a pathogen such as Botrytis cinerea, a grey rot agent (Jeandet et al., 1995; Bavaresco et al., 1997), Plasmopara viticola, a mildew agent (Dercks and Creasy, 1989) or Phomopsis viticola, which is responsible for excoriosis (Hoos and Blaich, 1990).
abiotic elicitation by environmental factors such as UV, temperature, light, asphyxia, natural agents extracted from other plants (Jeandet et al., 1997; Langcake and Pryce, 1977b; Douillet-Breuil et al., 1999), aluminium chloride (Adrian et al., 1996) or ozone (Sarig et al., 1996).

On elicitation, phytoalexins such as trans-resveratrol, trans-piceid, ε-viniferin and pterostilbene can be induced in leaves and berries (Soleas et al., 1997). This property of the de novo biosynthesis of phytoalexins in response to a stress, particularly after attack by a pathogen, suggests that these molecules could play the role of natural means of defence of the plants.

This role of defence molecules is corroborated by certain studies which seem to indicate a close interrelationship between the level of natural resistance of the plant and its ability to synthesise these molecules. For example, Langcake and McCarthy (1979) demonstrated a relation between the resistance of certain species of the Vitis kind to Botrytis cinerea or Plasmopara viticola and their capacity for the biosynthesis of phytoalexins (resveratrol and viniferin). Moreover, Dercks and Creasy (1989) showed that species resistant to Plasmopara viticola produce five times more phytoalexins than do sensitive species. Similarly, within the Vinifera species there are some vines which are more or less tolerant to attack by fungi depending on their capacity for producing phytoalexins.

Cell elicitation can be effected by means of agents or by means of various stresses, such as pressure, depressurisation, vacuum, pressure variations, the presence of a gas, a variable atmosphere, temperature, cold, light, cycle of brightness, radiation, a toxin, a plant toxin, agitation, a bacterium, a virus, fungi, a microorganism, ultrasound, IR, UV, asphyxia, etc.

Any method of elicitation known to one skilled in the art can be used to prepare a comminuted product which can be used in the composition according to the invention.

Thus, the comminuted products which can be used according to the invention can take any known form. Aqueous comminuted products and alcoholic comminuted products should be cited in particular, especially ethanolic or aqueous alcoholic comminuted products.

According to the invention, the comminuted product is preferably an aqueous comminuted product or a dry or substantially dry comminuted product.

The comminuted product can advantageously be freeze-dried in a subsequent stage. According to one advantageous embodiment, the comminuted product is a comminuted product of cells in their culture medium or in an extract of culture medium, said comminuted product advantageously being dried or freeze-dried afterwards.

The invention therefore relates to a cosmetic composition containing at least one dedifferentiated plant cell medium, characterised in that said medium is a comminuted product of dedifferentiated plant cells which are cultivated in an in vitro culture medium and elicited in the in vitro culture medium, said comminuted product being dispersed in said composition or being in a form suitable for being dispersed in said composition. The plant cells are preferably cultivated in an in vitro culture medium and are preferably elicited in an in vitro culture medium in order to synthesise at least one phytoalexin, said elicitation advantageously being effected after an in vitro culture stage of the plant cells without or substantially without elicitation in order to synthesise at least one phytoalexin. The composition contains at least one comminuted product of dedifferentiated plant cells which are elicited in a culture in vitro in order to synthesise at least one phytoalexin, this elicitation in order to synthesise at least one phytoalexin advantageously being effected after an in vitro culture stage of plant cells without elicitation, wherein said comminuted product containing at least one phytoalexin comprises at least 95%, advantageously at least 97%, preferably at least 99% by weight of the entirety of the dry materials derived from comminuted, dedifferentiated plant cells which are elicited in vitro, said comminuted product being dispersed in said composition or being in a form capable of be dispersed in said composition.

Substantially all, or even the entirety of the dry materials are derived from the comminuted, dedifferentiated plant cells which are elicited in vitro.

If the cells are washed to eliminate any culture medium present, it is possible without affecting the membrane structure of the cells to obtain a comminuted product of dedifferentiated elicited cells which does not contain a culture medium (less than 0.1% by weight based on the weight of the comminuted cells, for example). The comminuted product of dedifferentiated plant cells which are elicited in vitro advantageously comprises particles derived from vacuoles, particles derived from cytoplasm and particles derived from pecto-cellulose membrane, said comminuted product containing at least 0.1% by weight of phytoalexin(s).

For example, the composition may contain 0.005 to 25% by weight, advantageously 0.005 to 5% by weight, of a medium or of a comminuted product of dedifferentiated plant cells, said weight being calculated in dry form.

The composition advantageously contains a dry or substantially dry comminuted product of dedifferentiated plant cells which are elicited in vitro. The comminuted product is preferably a dry or substantially dry comminuted product of dedifferentiated plant cells which are elicited in an in vitro culture medium, said dry or substantially dry comminuted product having a water content less than 25% by weight, advantageously less than 15% by weight, preferably less than 10% by weight. In particular, said dry or substantially dry comminuted product contains an amount of water sufficient to ensure the integrity of the cell membrane after the comminution stage.

The average particle size of the solid particles of the comminuted product is advantageously less than 100 μm, more advantageously less than 10 μm, preferably less than 1 μm. The particle size distribution of the comminuted product is advantageously such that 90% by weight of the particles have a particle size ranging from the average particle size −25% to the average particle size +25%.

According to one particular embodiment, said comminuted product of dedifferentiated plant cells which are elicited in vitro contains at least one phytoalexin synthesised by the elicitation of dedifferentiated plant cells in an in vitro culture medium, or a mixture of phytoalexins such as these.

According to an advantageous feature of one embodiment, said comminuted product of dedifferentiated, elicited plant cells is a comminuted product of dedifferentiated plant cells which are elicited by an agent in the in vitro culture medium, said comminuted product being substantially free from said agent after elicitation in the in vitro culture medium.

According to another advantageous feature of one embodiment, said comminuted product of dedifferentiated, elicited plant cells is a comminuted product of dedifferentiated plant cells which are elicited by an agent in the culture medium, said agent being an agent, the presence of which is desired in the cosmetic composition, such as a surfactant, a dispersing agent, an acid, etc.

According one preferred embodiment, the dedifferentiated cells are elicited in an in vitro culture medium by a volatile agent, particularly a gas such as CO₂.

According one preferred embodiment, the dedifferentiated cells are elicited in an in vitro culture medium by a physical agent, particularly temperature, light, electromagnetic fields, UV, pressure or asphyxia.

According to a detail of one embodiment, said comminuted product of dedifferentiated plant cells which are elicited in an in vitro culture medium contains at least one terpenic, tannic or polyphenolic compound, said compound being synthesised by the in vitro elicitation of the dedifferentiated plant cells in their culture medium.

The comminuted product of dedifferentiated plant cells which are elicited in an in vitro culture medium advantageously exists in the form of a viscous suspension or a gel or of a substantially dry comminuted product, said suspension, gel or comminuted product being in a form which can be dispersed in the composition.

According to one particular embodiment, the comminuted cell product is a comminuted product of dedifferentiated vine cells which are elicited in an in vitro culture medium.

According to a detail of one preferred embodiment, the composition contains a comminuted product of dedifferentiated cells which are cultivated and elicited in their in vitro culture medium.

According to one advantageous embodiment, the composition contains a medium comprising dedifferentiated cells which are elicited in an in vitro culture medium, particularly a comminuted product of dedifferentiated, elicited cells, said medium or comminuted product containing at least 0.1% by weight of stilbenes based on the dry weight of the cells, particularly at least 0.2% by weight of stilbenes based on the dry weight of the cells, preferably at least 0.5% by weight of stilbenes based on the dry weight of the cells.

In the composition according to the invention, the comminuted product is derived from the culture of dedifferentiated plant cells, which are elicited in an in vitro culture medium and then dried of the species comprising Salvia, Coleus, Rosmarinus, Ginkgo, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry (prunus serrulata, prunus serrulata Kanzan), sequoia, chlorophytum, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, betula, tea, and mixtures of cells of such species.

According to one possible embodiment, the composition according to the invention comprises dedifferentiated, elicited plant cells which are comminuted (with a particle size less than 5 μm, for example), and dedifferentiated, elicited plant cells which are not comminuted or which are comminuted more coarsely, the dedifferentiated, elicited plant cells which are not comminuted or which are comminuted more coarsely being identical to or different from the comminuted, elicited cells and/or being elicited differently to the comminuted, elicited cells. According one particular embodiment, the dedifferentiated, elicited plant cells are separated into two distinct fractions, the first being subjected to fine comminution whilst the second is subjected to coarse comminution or is not comminuted.

According to a further object of the invention, the elicited dedifferentiated plant cells (elicited in vitro) are not comminuted, but submitted to a permeabilization or perforation step, such as a chemical permeabilzation or perforation step (with a chemical agent, such as saponin), a physical perforation or permeabilisation step, such as by electroporation.

The invention also relates to a method of preparing a composition for topical use according to the invention. In this method, the dedifferentiated plant cells are placed in an in vitro culture medium so as to enable cell growth, said dedifferentiated plant cells are elicited in their in vitro culture medium during a period of time sufficient for the synthesis of a sufficient quantity of metabolites, particularly at least one secondary metabolite, preferably at least one phytoalexin or a mixture of phytoalexins, and at least one medium or comminuted product of plant cells elicited from the culture medium is mixed with one or more excipients in order to prepare a cosmetic composition for topical use, particularly for cosmetic use, for example for the treatment of skin, hair, leather, nails, etc. The elicited cells are comminuted before and/or after their admixture with one or more excipients of the composition. Although it is possible to subject the dedifferentiated cells which are elicited in an in vitro culture medium to comminution, it is advantageous to separate the dedifferentiated cells which are elicited from the culture medium whilst not substantially affecting the cell membranes and then to subject said cells to comminution, advantageously after one or more washing stages which do not substantially affect the cell membranes and/or after one or more drying stages.

According to one particular embodiment, the dedifferentiated plant cells are subjected successively to in vitro culture stages without elicitation and to in vitro culture stages without elicitation.

The elicited are advantageously separated from the in vitro culture medium and the extract is subjected to drying, followed by comminution.

Although it is possible to freeze-dry the separated, washed, dedifferentiated, elicited cells without affecting the structure of the cell membranes and subsequently to comminute the freeze-dried product, the dedifferentiated, elicited cells which are separated and washed without affecting the structure of the cell membranes are advantageously subjected to controlled drying so as not substantially to affect the membrane barrier (drying temperature less 50° C., water content of the cells at least 3%, for example from 5 to 15%), and then to comminution. This enables the cells (membranes, cytoplasm and vacuoles) to be broken down during the comminution stage in order to ensure the release of phytoalexins during this stage.

The comminution stage and/or the drying stage (particularly a controlled drying stage) and/or the washing stage and/or the dedifferentiated cell separation stage are advantageously effected in the presence of one or more antioxidants agents such as vitamin E, etc., in order to prevent or to reduce any oxidation of one or more compounds from the cells (from the membrane, for example).

The cells are preferably elicited in their in vitro culture medium by means of an agent which after the extraction of the elicited cells is not present in the comminuted product of elicited cells.

In the method according to the invention, the stage of eliciting said dedifferentiated plant cells in their in vitro culture medium is advantageously controlled in order to obtain a medium containing at least 0.1% by weight of stilbenes based on the dry weight of the dedifferentiated cells, particularly at least 0.2% by weight of stilbenes based on the dry weight of the cells, preferably at least 0.5% by weight of stilbenes based on the dry weight of the cells.

The medium or comminuted product is derived, for example, from the culture of dedifferentiated plant cells, which are elicited in an in vitro culture medium and then advantageously dried, of species comprising Salvia, Coleus, Rosmarinus, Gingko, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry (prunus serrulata, prunus serrulata Kanzan), sequoia, chlorophytuni, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, betula, tea, and mixtures of cells of such species.

An example of the preparation of a comminuted product which can be used according to the invention is also given in the Examples.

The amount of comminuted product present in the composition according to the invention depends, of course, on the sought-after effect and can therefore vary to a large extent. To quote an order of magnitude, a comminuted product as defined previously can be used in an amount which constitutes from 0.01% to 20% of the total weight of the composition, preferably in an amount which constitutes from 0.1% to 5% of the total weight of the composition.

The comminuted product of dedifferentiated plant cells which are elicited in an in vitro culture medium, particularly in order to promote the production of one or more phytoalexins, can be used, for example, as an antioxidant, as an anti-radical agent, as a soothing agent, as an anti-irritation agent, or as a free radical scavenger.

In one particular aspect, the method of the invention enables a comminuted product to be obtained which is enriched in flavonoids such as flavanols, anthocyans and flavonols.

In another particular aspect, the method of the invention enables a comminuted product to be obtained which is enriched in non-flavonoid compounds such as phenol acids and derivatives of benzoic acid and also in original compounds such as stilbenes, trans- and cis-isomers of resveratrol and glucosides thereof, and trans- and cis-piceids.

In another particular aspect of the invention, the comminuted product which is enriched in polyphenols, phytoalexins and secondary metabolites retains the important pharmacological effects thereof. Thus, the comminuted products of the invention has one or more activities selected from antioxidant, anti-radical, anti-inflammatory, anti-proliferative, relaxant and vascular activities, etc.

In contrast to primary metabolites, secondary metabolites such as polyphenols accumulate in vivo in the plant in small amounts. In one aspect of the invention, in order to obtain these secondary metabolites in considerable amounts and in a continuous, stable manner, we have stimulated the biosynthesis thereof in cultures of standardised cells.

The dedifferentiated plant cells which can be used in the composition according to the invention can originate from all known species of plants. In this respect, mention should be made of genera such as Salvia, Coleus, Rosmarinus, Gingko, Cannabis, Colchicum, Gloriosa, Asparagus, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry (prunus serrulata, prunus serrulata Kanzan), sequoia, chlorophytum, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, betula, tea, etc.

According to the invention, use is made in particular of dedifferentiated plant cells derived from plants of the genera sequoia, vine, arganier, Cacao and chlorophytum, and of combinations thereof.

The comminuted products of dedifferentiated plant cells which can be used in the composition according to the invention can of course originate from mixtures of dedifferentiated plant cells obtained from different plant genera and/or obtained from different plant material, said cells being elicited in the same in vitro culture medium or in different in vitro culture media (to effect different elicitations, for example).

The expression “composition for topical use” should be understood to mean creams, ointments, lotions, suspensions, sticks, shampoos, gels, solutions (applicable by spraying, for example). For example, the composition for topical use can be a cosmetic composition, a dermatological composition, a skin hygiene composition, a perfume, etc.

According to the invention, the composition is preferably a cosmetic, particularly a composition for topical application.

The present invention further relates to a method for the cosmetic treatment of skin, characterised in that a composition according to the invention, comprising at least one comminuted product of dedifferentiated plant cells which are elicited in an in vitro culture medium, freeze-dried and incorporated or dispersed in a cosmetic product, is applied to the skin, hair, and/or mucous membranes.

In particular, the cosmetic treatment method of the invention can be put into effect by applying the cosmetic compositions as defined above by the customary technique for the use of these compositions. For example: the application of creams, gels, serums, lotions, milks, shampoos or sun-reflective compositions to the skin.

While the invention discloses here above relates to compositions containing comminuted dedifferentiated plant cells which were elicited in vitro, to method for the preparation or use of such compositions, etc., the inventions relates furthermore to such compositions, methods, etc. in which the dedifferentiated cells which are elicited are permeabilized, for example perforated, elctroperforated, chemically perforated, said permeabilized elicited dedifferentiated plant cells replacing the comminuted elicited dedifferentiated plant cells.

Details of such compositions, methods, etc. with permeabilized elicited deddifferentiated cells are given here after.

An object of the invention is a composition for topical application containing at least elicited dedifferentiated plant cells, whereby said dedifferentiated plant cells are elicited in vitro in a culture in order to synthesise at least one phytoalexin, whereby the elicited dedifferentiated plant cells are permeabilized and at least in a form suitable for being dispersed in said composition. Advantageously, the permeabilized elicited dedifferentiated plant cells (cells which are rendered permeable, i.e. in the membrane of which pores or openings have been made) are dispersed in said composition.

Preferably the dedifferentiated plant cells are elicited in vitro in a culture in order to synthesise at least one phytoalexin, after an in vitro stage of plant cell culture without elicitation.

Most preferably, the composition is a cosmetic composition.

According to an embodiment, the permeabilized elicited dedifferentiated plant cells are electro pored cells, i.e. submitted to an electroporation with an AC and/or DC current, with a voltage for example lower than 50V, such as comprised between 1 and 10V. When using pulsed current, the duration of the electrical pulses can be less than 200 μs. The current density will be selected so as to avoid an excessive increase of temperature. Electroporation is preferred as no agent is added to the composition, i.e. no impurities remain in the composition.

According to a specific detail, the permeabilized elicited dedifferentiated plant cells are at least partially dried, most preferably substantially completely dried before permeabilized.

According to an embodiment, the composition contains 0.005 to 25%, advantageously 0.005 to 5% by weight of a permeabilized elicited dedifferentiated plant cells, said weight being calculated in dry form.

According to another embodiment, the composition contains a substantially dry permeabilized elicited dedifferentiated plant cells which are elicited in vitro, said substantially dry plant cells having a water content less than 25% by weight, advantageously less than 15% by weight, preferably less than 10% by weight.

According to a detail, the permeabilized dedifferentiated plant cells which are elicited in vitro contains at least one phytoalexin synthesised by the in vitro elicitation of dedifferentiated plant cells.

Preferably, said permeabilized elicited dedifferentiated plant cells is dedifferentiated plant cells which are elicited in vitro by an agent in the culture medium before being permeabilized, whereby said dedifferentiated plant cells are substantially free from said agent after elicitation. For example, the dedifferentiated cells are elicited in vitro by a volatile agent.

More specifically, the dedifferentiated plant cells which are elicited in vitro contains at least one compound selected from the group consisting of terpenic compounds, tannic compounds, polyphenolic compounds and mixtures thereof, said compound being synthesised by the in vitro elicitation of the dedifferentiated plant cells in their culture medium.

According to an embodiment, the dedifferentiated plant cells which are elicited in vitro and permeabilized are in a form selected from the group consisting of viscous suspensions, gels and substantially dry powders.

According to a specific embodiment, the composition comprises dedifferentiated vine cells which are elicited in vitro and permeabilized.

Advantageously, the composition contains dedifferentiated plant cells which are cultivated and elicited in their in vitro culture medium, and submitted to a permeabilizing step.

Preferably, the composition is substantially free from culture medium.

According to a further detail, the composition comprises permeabilized elicited dedifferentiated cells which are elicited in vitro containing at least 0.1%, advantageously at least 0.2%, preferably at least 0.5% by weight of stilbenes based on the dry weight of the dedifferentiated, elicited cells.

Most preferably, the permeabilized dedifferentiated, elicited plant cells is derived from the culture of dedifferentiated plant cells, which are elicited and permeabilized, of at least one species selected from the group consisting of Salvia, Coleus, Rosmarinus, Gingko, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, betula, tea and mixtures of cells of such species.

According to further embodiment, the composition comprises at least one excipient and/or at least a glycol ester and/or at least propylene glycol.

According to a detail, the permeabilized elicited dedifferentiated plant cells are provided with a coating, such as a coating of an glycol ester or propylene glycol.

While in the composition comprising permeabilized elicited plant cells, said cells are not comminuted, in some special cases, it can be advantageous to further comminute the permeabilized cells.

The composition can also comprise a mixture of comminuted elicited dedifferentiated plant cell and of permeabilized elicited dedifferentiated plant cell not submitted to a comminution.

The invention relates thus also to a method of preparing a composition for topical use containing at least one excipient and at least one permeabilized elicited dedifferentiated plant cells, whereby said dedifferentiated plant cells are elicited in vitro in a culture in order to synthesise at least one phytoalexin, whereby the permeabilized elicited dedifferentiated plant cells are at least in a form suitable for being dispersed in said composition, in which:
dedifferentiated plant cells are placed and growth in an in vitro culture medium,
the dedifferentiated plant cells are elicited in their culture medium during a period of time sufficient for the synthesis of elicited dedifferentiated plant cells with a sufficient quantity of metabolites, and
the dedifferentiated plant cells elicited in the in vitro culture medium are mixed with one or more excipients in order to prepare a cosmetic composition
   whereby the elicited dedifferentiated plant cells are permeabilized in at least one permeabilizing step selected from the group consisting of permeabilizing step before mixing the elicited dedifferentiated plant cells with at least one excipient, permeabilizing step after mixing the elicited dedifferentiated plant cells with at least one excipient, permeabilizing step after a drying stage, and combinations thereof.

Advantageously, said plant cells which are elicited in vitro are subjected to a filtration step, before being permeabilized.

According to an embodiment, the dedifferentiated plant cells are elicited in their in vitro culture medium by means of an agent which, after extracting the elicited cells from the culture medium whilst retaining the membrane structure of the cells, does not occur in the elicited cells.

According to another embodiment, the dedifferentiated plant cells are placed in a growth culture in vitro, are elicited in said in vitro culture medium, are at least partly separated from the growth culture, are then permeabilized and dispersed in a composition for the treatment of the human body.

According to a detail, the elicited dedifferentiated plant cells are at least submitted to one washing step before being permeabilized. Preferably, the cells are at least partly dried before being permeabilized.

According to still another embodiment, the dedifferentiated plant cells are elicited in their culture medium by an eliciting agent which does not form an impurity in the permeabilized dedifferentiated and elicited cells and/or for the composition.

Preferably, the dedifferentiated plant cells are at least one species selected from the group consisting of Salvia, Coleus, Rosmarinus, Gingko, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, cominiphora wighii, eucalyptus punctata, lavandula angustifolia, citrus limon, vanilla planifolia, marrubium vulgare, pilocarpus jaborandi, roses, betula, tea, and mixtures thereof.

A further subject matter of the invention is a permeabilized product of dedifferentiated plant cells which are elicited in an in vitro culture medium and thereafter permeabilized, wherein said permeabilized product containing at least one phytoalexin, said permeabilized product being in a form suitable for being dispersed in a composition selected from the group consisting of cosmetic compositions and pharmaceutical compositions.

The product is free from elicitation agent and/or free from culture medium.

More specifically, the product contains at least 0.1%, advantageously at least 0.2%, preferably at least 0.5% by weight of stilbenes based on the dry weight of the cells.

According to a specific embodiment, the product comprises a glycol ester, especially propylene glycol. The product is for example a suspension of cells in ester glycols, such as propylene glycol.

The product comprises advantageously from 5 to 30% by weight of permeabilized or perforated dedifferentiated plant cells elicited in vitro.

Preferably, the dedifferentiated plant cells are selected from the group of species consisting of Salvia, Coleus, Rosmarinus, Ginkgo, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, betula, tea, and mixtures thereof.

Still a further object of the invention is a composition for topical application containing at least one permeabilized product of in vitro elicited dedifferentiated plant cells containing at least one phytoalexin, whereby said permeabilized product (for example perforated) contains at least 0.1%, advantageously 0.2%, preferably 0.5% by weight of stilbenes based on the dry weight of the cells.

Preferably, said permeabilized product is a permeabilized product of dedifferentiated plant cells, which are elicited in vitro, the dedifferentiated plant cells being selected from the group of species consisting of Salvia, Coleus, Rosmarinus, Ginkgo, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, betula, tea, and mixtures thereof. The composition is for example a cosmetic composition.

Essential features of compositions or products according to the invention, of methods according to the invention, of comminuted products according to the invention and of composition with perforated/permeabilised cells are given in the claims. The following examples of compositions illustrate the invention without by any means limiting it. In the compositions, the proportions given are percentages by weight.

In these examples, a preferred method was used as defined in summary below:

Description of a Preferred Method of Obtaining a Comminuted Product

Stage 1: Preparation of dedifferentiated cells cultivated in an in vitro culture medium
Stage 2: Elicitation of the dedifferentiated cells in the culture medium
Stage 3: Extraction of the dedifferentiated cells which are elicited in an in vitro culture medium, for example by filtration of the culture medium followed by one or more washing stages which are conducted in particular so as not to destroy the structure of the cell membranes. The cells are advantageously washed in order substantially to eliminate any trace of culture medium, said washing being conducted so as not to destroy the structure of the cell membranes.
Stage 4: Drying or freeze-drying the dedifferentiated cells which are elicited in an in vitro culture medium, this drying operation advantageously being conducted so as not to destroy the structure of the cell membranes.

This stage is advantageously conducted at a temperature less than 60° C., for example between −60° C. and 50° C.
Stage 5: comminution (stage 5 is advantageously performed; in some cases, however, this stage can prove not to be necessary). The comminuted product thus contains substantially all the dry components which form the cell, i.e. substantially all the components of the membrane, of the cytoplasm and of the vacuoles.
Stage 6: admixture and/or incorporation of one or more excipients and/or of other active constituents (particularly from other cells/comminuted plant materials) for the preparation of the composition for topical use
   Description of a Preferred Method of Obtaining a Permeabilized Cell Containing Product
Stage 1: Preparation of dedifferentiated cells cultivated in an in vitro culture medium
Stage 2: Elicitation of the dedifferentiated cells in the culture medium
Stage 3: Extraction of the dedifferentiated cells which are elicited in an in vitro culture medium, for example by filtration of the culture medium followed by one or more washing stages which are conducted in particular so as not to destroy the structure of the cell membranes. The cells are advantageously washed in order substantially to eliminate any trace of culture medium, said washing being conducted so as not to destroy the structure of the cell membranes.
Stage 4: Possible drying or partial drying the dedifferentiated cells which are elicited in an in vitro culture medium, this drying operation advantageously being conducted so as not to destroy the structure of the cell membranes.
Stage 5: electroporation and/or chemical poration (with saponin) of the cells.
Stage 6: possible incorporation of the perforated cells in a glycol ester compostion, preferably in propylene glycol.
Stage 7: possible further admixture and/or incorporation of one or more excipients and/or of other active constituents (particularly from other cells/comminuted plant materials) for the preparation of the composition for topical use

### PREFERRED EXAMPLES

### Example 1

### Production of a Comminuted Product of Dedifferentiated Vine Cells Elicited In Vitro

The first step for the development of plant cell cultures consists of selecting the plant which produces the sought-after substances. It is nowadays acknowledged that within the same species there is a variability of the production capacities for a given metabolite, part of which variability is of genetic origin. When it is possible, it is therefore necessary to exploit this variability by selecting the best genotype, i.e. the one which is the most productive for the sought-after metabolite. Primary proliferations can successfully be induced from sterilised fragments of a selected plant organ (leaf, stem, root, etc.), placed in vitro on a solid medium (gelose). Thus, after some weeks in culture, undifferentiated accumulations of cells termed calluses are formed in the explants. The growth of these calluses is maintained by successive subculturing stages on a new nourishing medium. These conditions of culture induce the spontaneous appearance of morphological and metabolic variability between calluses derived from the same plant or the same explant. However, the maintenance of constant environmental conditions tends to reduce this variability. Thus, after one to two years of regular subculturing, a collection of stable strains is obtained which exhibit the growth and production characteristics of very different metabolites.

At this stage, it is then possible, with the help of well defined tests, to select the strain or strains which produce a significant amount of the compounds of interest. Introducing these calluses into a liquid environment then enables progress to be made move towards larger production volumes, firstly in 250 ml phials, and subsequently in a bio-reactor (20 litres or more). The cell suspensions thus obtained, which are formed from aggregates and isolated cells, can again exhibit heterogeneity (somaclonal variability). An additional selection is then made in order to obtain highly productive cell lines. In addition to this cloning operation, the production of the metabolite of interest can also be optimised by modifying the culture conditions, resulting in the development of media termed production media. This medium is identical to the cell subculture medium except for the concentration of sucrose, which is multiplied by two. During their culture in a production medium, highly productive Cabernet Sauvignon vine cell lines are elicited ten days after inoculation, by 254 nm UV light from a Wilber-Lourmat T-30C lamp (600 μW/m²) placed at a distance of 1 m to provide direct illumination of the cells for 10 minutes, which induces a considerable accumulation of polyphenols, particularly stilbenes, in the cells. This means of elicitation clearly does not form any impurity in the cell culture. At the end of the culture stage, i.e. three days after elicitation, the plant cells are filtered to remove the remaining culture medium and are rinsed in cold water (4° C.). A fresh biomass of about 350 grams per litre of culture is thus obtained. The extracted cells are then dried and comminuted. Drying is effected to a more or less considerable extent in order to obtain a comminuted product of dedifferentiated vine cells, which are elicited in vitro, as a viscous suspension or a gel or as a substantially dry powder. By controlling the rate of drying and the water content of the cells, which is advantageously between 3 and 10%, it is possible not to destroy the cell membrane before the comminution stage, which enables the phytoalexins contained in the cells, particularly in the vacuoles of the cells and/or in the membrane, to be released during the comminution stage. In the case of substantially dry cells, about 20 grams of dry biomass per litre of culture are obtained after freeze-drying in a Virtis apparatus (Uni-Trap 10-100). The powder obtained after comminution in a Mortier-type mill is light, ultrafine (particle size less than 10 μm), beige in colour, and clear.

The elicited, dried cells are comminuted to form particles having an average particle size (by weight) between 1 and 10 μm. If necessary, it is possible to subject the comminuted product to particle size separation (sifter, etc.) so as to retain only particles of a given particle size, for example within the ranges 5-10 μm, 1-5 μm, less than 3 μm, etc.

Dedifferentiated cells were prepared from various materials and were elicited by means of various agents. The data are summarised in the following Table:

| | Material from which | |
|---|---|---|
| | the dedifferentiated | |
| Example 1 (vine) | cells originate | Type of elicitation |
| 1A | branch less than one | UV radiation for 3 |
| | year old | days |
| 1B | cuticle of ripe grape | carbon dioxide for 24 |
| | | hours |
| 1C | cuticle of green grape | carbon dioxide for 2 |
| | | days |
| 1D | grape seed | UV radiation and |
| | | carbon dioxide for 5 |
| | | days |
| 1E | root | UV radiation for 12 |
| | | hours |
| 1F | green leaf | UV radiation for 15 |
| | | hours |
| 1G | bud | UV radiation for 75 |
| | | hours |
| 1H | residue from a | UV radiation for 2 days |
| | pressing stage | |
| 1I | residue from a | UV radiation for 3 days |
| | pressing stage | |

It should also be noted that comminution of the dedifferentiated, elicited cells is advantageously effected in the presence of one or more agents or excipients of the cosmetic composition in order to ensure the release of phytoalexins in at least some agents of the cosmetic composition.

### Example 2

### Determination by HPLC of the Stilbene Content of Elicited (Example 1A) and Non-Elicited Vine Cells

Materials and Methods:

Bischoff Model 2,200 pump
automatic injector (Alcoot Model 788 autosampler)
Ultrasep C18 column (30×cm 0.18 cm); porosity 6 mm
Jasco 821-FI fluorescence detector.

Fluorescence was detected with excitation at 300 nm and emission at 390 nm. The eluant used was composed of methanol: water, 40:60 (v/v), the pH of which was adjusted to 8.3 with 1M KOH.

Results:

The stilbene content was about 1% based on the weight of dry matter of the vine cells elicited in vitro. The phytoalexin content remained less than 0.05% in non-elicited cells. This increased stilbene content (20-fold) is a means of controlling of the elicitation stage, and therefore of the manufacturing process of a comminuted product according to the invention. On account of the high concentration of stilbene in the comminuted product according to the invention, it is possible, by extraction, distillation, or crystallisation operations, etc., to produce media which are even more concentrated in stilbenes or phytoalexins, or even to produce substantially pure photoalexins.

The compositions according to the invention therefore comprise a high ratio of phytoalexin content to comminuted cell membrane content compared with the ratio of phytoalexin content to comminuted cell membrane content in non-elicited or natural plant cells.

### Example 3

### Pharmacological Activity of a Comminuted Vine: Antioxidant

The anti-radical activity of the product obtained according to Example 1A was investigated in vitro. A SKINETHIC® reconstituted model epidermis was used, which enabled this activity to be revealed by the determination of malondialdehyde (MDA) after the induction thereof by ultraviolet B radiation.

Epidermis Distribution

The test was performed in triplicate, after the product (the comminuted product from Example 1A) had been in contact with the epidermises for 24 hours. Keratinocytes of human origin were seeded on 0.63 cm² polycarbonate filters in a defined medium (modified MCDB 153) and supplemented. The cells were cultivated for 14 days at the air/liquid interface, the culture medium being changed every other day.

The epidermises which were thus formed were used for carrying out the investigation from the 17th day of the culture.

SKINETHIC® Reconstituted Epidermis Batches:
Batch 1: 3 control epidermises which received neither product nor irradiation
Batch 2: 3 epidermises exposed to UVB (150 mJ/cm²)
Batch 3: 3 epidermises treated with SOD+Catalase+UVB (150 j/cm²)
Batch 4: 3 epidermises treated with comminuted product (0.1%)
Batch 5: 3 epidermises treated with comminuted product (0.5%)
Batch 6: 3 epidermises treated with comminuted product (1%)
Batch 7: 3 epidermises treated with comminuted product (0.1%)+UVB (150 mJ/cm²)
Batch 8: 3 epidermises treated with comminuted product (0.5%)+UVB (150 mJ/cm²)
Batch 9: 3 epidermises treated with comminuted product (1%)+UVB (150 mJ/cm²)
   Determination of Malondialdehyde (MDA): Index of Lipoperoxidation
   Extraction of Malondialdehyde (MDA)

24 hours after the treatment of the SKINETHIC® reconstituted epidermises, the cells were placed in suspension in:
250 μl of Tris buffer, 50 mM, pH 8, containing 0.1M NaCl, 20 mM EDTA
25 μl of 7% SDS
300 μl of HCl (0.1N)
38 μl of 1% phosphotungstic acid in water
300 μl of 0.67% thiobarbituric acid in water

After 1 hour of incubation in the dark at 50° C. and cooling in ice water, 300 μl of n-butanol was added to each tube. These were centrifuged at 10,000 g at 0° C. for 10 minutes. The supernatant phase was recovered for the determination of MDA.

Determination of Malondialdehyde (MDA)

MDA was determined by measuring the fluorescence after separation of the MDA-TBA complex by HPLC.
Bischoff Model 2,200 pump
automatic injector (Alcoot Model 788 autosampler)
Ultrasep C18 column (30×cm 0.18 cm); porosity 6 mm
Jasco 821-FI fluorescence detector.

Fluorescence was detected with excitation at 515 nm and emission at 553 nm. The eluant used was composed of methanol: water, 40:60 (v/v), the pH of which was adjusted to 8.3 with 1M KOH.

Quantification was effected by comparison with standards which were treated identically to the samples (0.125; 0.25; 0.5 and 1 μM) using ICS (Instrumentation Consommation Service) data processing software (Peak 3).

Determination of Proteins

The proteins were determined by the method of BRADFORD. The increase in absorbance at 595 nm is proportional to the concentration of the proteins, and was determined using a UNICAM 8625 spectrophotometer.

Results

Determination of Malondialdehyde (MDA) in the Cell Homogenate

The results are given in the Table below:

| | | Variation of MDA |
|---|---|---|
| | | (in %) in relation to |
| | MDA(μM/mg proteins) | the control |
| Control | 546 ± 40.81 | — |
| comminuted | 445.3 ± 57.72* | −18 (decrease) |
| product (0.1%) | | |
| comminuted | 409.5 ± 48.58* | −25 (decrease) |
| product (0.5%) | | |
| comminuted | 325.5 ± 28.85* | −40 (decrease) |
| product (1%) | | |

*significantly different from the control: p < 0.005 (Wilcoxon Rank Sum Test).

The results obtained showed that there was a significant protection of the comminuted product from physiological lipoperoxidation, namely 18%, 25% and 40% at dilutions of 0.1; 0.5 and 1%, respectively.

Induced Lipoperoxidation

The results are given in the Table below:

| | | Variation of MDA |
|---|---|---|
| | | (in %) in relation to |
| | MDA | the control with |
| | (μM/mg proteins) | UVB |
| Control | 546 ± 40.81 | — |
| UVB (150 mJ/cm²) | 792.4 ± 59.4 | (increase of |
| | | 45% in relation to the |
| | | control without UVB) |
| SOD/Catalase + | 482.5 ± 22.1 | −39 (decrease) |
| UVB (150 mJ/cm²) | | |
| comminuted product (0.1%) + | 545 ± 43.4** | −31 (decrease) |
| UVB (150 mJ/cm²) | | |
| comminuted product (0.5%) + | 485.5 ± 35.6** | −39 (decrease) |
| UVB (150 mJ/cm²) | | |
| comminuted product (1%) + | 420.3 ± 46.3* | −47 (decrease) |
| UVB (150 mJ/cm²) | | |

*Significantly different from the control; p < 0.005 (Wilcoxon Rank Sum Test).
**Significantly different from the control;; p ≦ 0.05 (Wilcoxon Rank Sum Test).

The results obtained show that there is significant protection of comminuted product from lipoperoxidation induced by ultraviolet B radiation (150 mJ/cm²), namely 31%, 39% and 47% at concentrations of 0.1; 0.5 and 1% respectively, by comparison with SOD/catalase protective enzymes (39%).

Under the experimental conditions employed, the comminuted product appeared to exhibit significant antiradical activity in SKINETHIC® reconstituted epidermises after 24 hours of contact. This activity was revealed by the determination of MDA.

In fact, the MDA determinations show that at concentrations of 0.1; 0.5 and 1%, the comminuted product investigated:
significantly decreases the physiological MDA level by 18%, 25% and 40%, respectively.
significantly protects the cells from lipoperoxidation induced by ultraviolet B radiation (UVB 150 mJ/cm²) by decreasing the level of induced MDA by 31%, 39% and 47% compared with SOD-catalase, which decreases the level of MDA by 39%.

In conclusion, the comminuted product exhibits an anti-radical effect both under physiological conditions and under conditions of induction by ultraviolet B radiation. It is clear from this test that the comminuted product exhibits a significant anti-radical effect.

Taking into account the model employed (reconstituted epidermis), it can already be envisaged that the comminuted product can be employed in preparations for topical use, at a minimum active dose of 0.1%.

### Example 4

### Investigation of the Effect of the Product on Respiration Rate (Oxygen Consumption in Nanoatoms of Oxygen per Million Cells per Minute)

This experiment was performed under 2 different conditions:
Effect on the rate of basal cell respiration in non-permeabilised cells and in the presence of glucose, in order to evaluate cell respiration.
Effect on the rate of respiration of permeabilised cells in the presence of the respiratory substratum pyruvate-malate, in order to evaluate mitochondrial respiration.

This investigation was performed on human keratinocytes in a dissociated trypsin culture. 5 to 10 million keratinocytes in a culture were placed in suspension in 1 ml of Hanks-Hepes medium at 30° C. containing glucose (20 mM). Respiration was monitored in real time and was expressed in nanoatoms of oxygen consumed per minute per 10⁶ cells. The addition of different amounts of product to the cell of the Oxygraph instrument enabled any stimulation or inhibition of respiration to be detected.

The amount of oxygen dissolved in an incubation medium was determined using a Clark electrode. The oxygen which diffused through a Teflon film was reduced at the platinum cathode, which was polarised at −0.8 volt. Under these conditions, the current passing between this cathode and the silver anode was proportional to the oxygen concentration in the solution. A salt bridge was formed by a half-saturated KCl solution. The measurements were acquired and processed on a microcomputer.

Effect on Basal Cell Respiration Rate

This test was performed on whole, non-permeabilised cells in the presence of glucose.

Tests were carried out on a 3% master solution of the product.

The results are given in the Table below:

Rate of Basal Cell Respiration

| | | | | | | |
|---|---|---|---|---|---|---|
| Comminuted | 0.00 | 2 | 5 | 10 | 50 | 100 |
| product | | | | | | |
| (μl of product) | | | | | | |
| Basal | 1.15 ± 0.5 | 1.22 ± 0.21 | 1.31 ± 0.14 | 1.58 ± 0.64 | 2.30 ± 0.27 | 2.58 ± 0.21 |
| respiration | | | | | | |
| (natoms/minute/ | | | | | | |
| 10⁶ cells) | | | | | | |
| (n = 3) | | | | | | |
| % of basal cell | 100 | 106 | 114 | 137 | 200 | 224 |
| respiration | | | | | | |

Effect on Mitochondrial Respiration Rate

This test was performed on permeabilised cells in the presence of the pyruvate-malate respiratory substrate.

The results are given in the Table below:

Rate of Mitochondrial Respiration in the Presence of Pyruvate-Malate

| | | | | | | |
|---|---|---|---|---|---|---|
| Comminuted | 0.00 | 2 | 5 | 10 | 50 | 100 |
| product | | | | | | |
| (μl of product) | | | | | | |
| Pyruvate | 1.58 ± 0.12 | 1.98 ± 0.05 | 2.05 ± 0.14 | 2.25 ± 0.42 | 2.58 ± 0.22 | 3.02 ± 0.38 |
| respiration | | | | | | |
| (natoms/minute/ | | | | | | |
| 10⁶ cells) | | | | | | |
| (n = 3) | | | | | | |
| % of pyruvate | 100 | 125 | 130 | 142 | 163 | 191 |
| respiration | | | | | | |

The results show that the comminuted product, in different doses, increases the rate of respiration (oxygen consumption) both in whole, non-permeabilised cells (in the presence of glucose), resulting in an increase in basal cell respiration, and in permeabilised cells (in the presence of pyruvate-malate), resulting in an increase in mitochondrial respiration.

### Example 5

### Investigation of the Effect of the Product on the Rate of Synthesis of PTA in nmol per Million Cells per Minute

This stage was effected under 2 different conditions:
Effect on the rate of synthesis of PTA in non-permeabilised cells and in the presence of glucose, to evaluate the rate of cell synthesis.
Effect on the rate of synthesis of PTA in permeabilised cells in the presence of pyruvate-malate respiratory substrate, to evaluate the rate of mitochondrial synthesis.

This investigation was performed on human keratinocytes in a dissociated trypsine culture. 5 to 10 million keratinocytes in culture were placed in suspension in 1 ml of Hanks-Hepes medium containing glucose (20 mM) at 30° C. The addition of different amounts of product to the vessel enabled any activation or inhibition of the rate of synthesis of PTA to be detected.

The amount of PTA present in the medium was determined by virtue of the following enzymatic reaction:

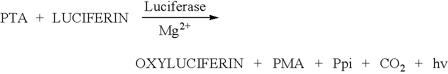

The reaction was effected in a device of the Luminoscan type, using the PTA monitoring reagent (PTA Bioluminescence Assay Kit HS II) supplied by Boehringer Mannheim.

The intensity of light emitted during this reaction was measured by a luminometer (Luminoscan) which recorded it in RLU (relative luminosity units). The measured RLUs were converted into mol PTA using a standard range of PTA as a reference.

Effect on the Rate of Synthesis of PTA: Rate of Basal Cell Synthesis

This test was performed on whole, non-permeabilised cells in the presence of glucose. The results are given in the Table below.

Rate of Synthesis of PTA

| | | | | | | |
|---|---|---|---|---|---|---|
| COMMINUTED PRODUCT | 0.00 | 2 | 5 | 10 | 50 | 100 |
| (μl product) | | | | | | |
| Rate of synthesis | 2.58 ± 0.15 | 2.65 ± 0.12 | 3.08 ± 0.22 | 3.12 ± 0.30 | 5.80 ± 0.95 | 5.95 ± 0.72 |
| (nmolesPTA/mn/10⁶ cells) | | | | | | |
| (n = 3) | | | | | | |
| % of cellular synthesis | 100 | 103 | 119 | 121 | 225 | 231 |

Effect on the Rate of Synthesis of Mitochondrial PTA

Rate of Synthesis of Mitochondrial PTA in the Presence of Pyruvate-Malate

| | | | | | | |
|---|---|---|---|---|---|---|
| COMMINUTED | 0.00 | 2 | 5 | 10 | 50 | 100 |
| PRODUCT | | | | | | |
| (μl product) | | | | | | |
| Rate of synthesis | 4.15 ± 0.22 | 4.95 ± 0.16 | 4.95 ± 0.18 | 5.02 ± 0.16 | 5.75 ± 0.28 | 7.58 ± 0.85 |
| (nmolesPTA/mn/10⁶ | | | | | | |
| cells) (n = 3) | | | | | | |
| % of mitochondrial | 100 | 119 | 119 | 121 | 139 | 183 |
| synthesis | | | | | | |

The results show that the comminuted product, in different doses, increases the rate of synthesis of PTA both in whole, non-permeabilised cells (in the presence of glucose), resulting in an increase the synthesis of cell PTA, and in permeabilised cells (in the presence of pyruvate-malate), resulting in an increase in mitochondrial synthesis.

### Example 6

Investigation of the effect of the product on the energy metabolism of cells in culture. Determination of cell adenylic nucleotides (PTA, ADP and PMA) in nmoles/mg_ of proteins, and calculation of the energy load (EL) of cells treated for 5 days by the product.

Human keratinocytes were placed in a culture for 5 days in the absence and in the presence of the product (10⁷ cells per measurement).

Once trypsinised, the cells were harvested and the concentrations of adenylic nucleotides were determined by HPLC.

This test was performed on whole, non-permeabilised cells.

The results are given in the Table below.

| Dose | [PTA] | [ADP] | [PMA] | [PTA/ADP] | Sum | E.L. |
|---|---|---|---|---|---|---|
| Control | 4222 | 1014 | 748 | 4.16 | 5984 | 0.79 |
| 0.02% | 4532 | 1435 | 837 | 3.16 | 6804 | 0.77 |
| 0.05% | 5292 | 1327 | 779 | 3.99 | 7398 | 0.80 |
| 0.1% | 6184 | 1231 | 796 | 5.02 | 8211 | 0.83 |
| 0.5% | 6848 | 2195 | 978 | 3.12 | 10021 | 0.79 |
| 1% | 7791 | 2532 | 1131 | 3.08 | 11454 | 0.79 |

The concentrations of PTA, ADP and PMA are expressed in nmol/mg proteins (n=3).
Sum=[PTA]+[ADP]+[PMA]
Energy load (E.L.)=[PTA]+½[ADP]/[PTA]+[ADP]+[PMA]

The results show that the comminuted product, in different doses, increases the following, depending on the dose:
the concentration of synthesised PTA,
the total concentration of cell adenylic nucleotides.

Moreover, the energy load (EL) remained constant, resulting in a stable energy equilibrium between the adenylic nucleotides:
PTA→ADP+Pi
PTA→PMA+PPi
(Pi: inorganic phosphate)

These results correlate perfectly with those obtained during the first 2 stages, and confirm that the product results in the stimulation of the cell energy metabolism.

### Example 7

### Investigation of the Tolerance of a Comminuted Vine Product

An investigation of cutaneous and ocular tolerance in vitro was performed as a preliminary measure with regard to the development of cosmetic preparations.

These tests revealed a perfect local tolerance (cutaneous and ocular) to the comminuted product at a concentration of 0.3%.

The comminuted cell products can be used directly to form cosmetic compositions for topical use.

Some non-limiting examples of compositions for topical use are given below.

### Example 8

### Dispersion of Elicited Whole Vine Cells in a Cosmetic Base

Vine cells were obtained as described in Examples 1A to 1I. The cells of these examples were used separately or in admixture for the preparation of a cosmetic composition. The cells were dispersed after freeze-drying, and without having been comminuted, in the following base:

| | |
|---|---|
| deionised water | 85.61% |
| mineral oil | 9.00% |
| cetyl alcohol | 3.00% |
| ceteareth-20 | 0.75% |
| vine cells | 0.20% |
| fragrance | 0.15% |
| carbomer | 0.10% |
| methylchloroisothiazoline | 0.065% |
| and methylisothiazoline | |
| [kathon CG] | |
| sodium hydroxide (45%) | 0.06% |
| butylated hydroxyanisole | 0.06% |
| TOTAL | 100.00% |

The composition obtained exhibited a homogeneous dispersion of the cells in the cream and a very fine particle size. A test for cleanliness showed the absence of germs and fungi as well as a remarkable stability of the composition. The result obtained from a transcutaneous investigation showed the passage of the active constituents, particularly polyphenols, through cutaneous tissue.

### Example 9

### Dispersion of Elicited, Comminuted Vine Cells in a Cosmetic Base

Vine cells were obtained as described in Examples 1A to 1I. The cells of these examples were used separately or in admixture for the preparation of a cosmetic composition. After freeze-drying, and comminution, the cells were dispersed in the following base:

| | |
|---|---|
| deionised water | 85.61% |
| mineral oil | 9.00% |
| cetyl alcohol | 3.00% |
| ceteareth-20 | 0.75% |
| vine cells | 0.20% |
| fragrance | 0.15% |
| carbomer | 0.10% |
| methylchloroisothiazoline | 0.065% |
| and methylisothiazoline | |
| [kathon CG] | |
| sodium hydroxide (45%) | 0.06% |
| butylated hydroxyanisole | 0.06% |
| TOTAL | 100.00% |

The composition obtained exhibited a homogeneous dispersion of the comminuted cell product in the cream and a very fine particle size. A test for cleanliness showed the absence of germs and fungi as well as a remarkable stability of the composition. The result obtained from a transcutaneous investigation showed the passage of the active constituents, particularly polyphenols, through cutaneous tissue.

### Example 10

### Dispersion of Elicited Whole Vine Cells in a Cosmetic Base

Vine cells were obtained as described in Examples 1A to 1I. The cells of these examples were used separately or in admixture for the preparation of a cosmetic composition. The cells were dispersed, after freeze-drying and without having been comminuted, in the following base:

| | |
|---|---|
| water | 46.89% |
| sodium lauryl sulphate (25%) | 36.40% |
| PEG-7 glyceryl cocoate | 2.00% |
| laureth-2 | 1.50% |
| laureth-11 sodium carboxylate | 4.00% |
| cocamidopropyl betaine & benzoic acid | 3.48% |
| sodium chloride | 1.60% |
| propylene glycol | 1.00% |
| fragrance | 0.13% |
| PEG-40 hydrogenated castor oil | 0.50% |
| & propylene glycol & water | |
| oleth-10 | 0.50% |
| sodium phosphate | 0.30% |
| disodium phosphate | 0.08% |
| citric acid (50%) | 0.52% |
| sodium benzoate | 0.50% |
| vine cells | 0.20% |
| salicylic acid | 0.20% |
| phenoxyethanol | 0.20% |
| TOTAL | 100.00% |

The composition obtained exhibited a homogeneous dispersion of cells in the cream and a very fine particle size. A test for cleanliness showed the absence of germs and fungi as well as a remarkable stability of the composition. The result obtained from a transcutaneous investigation showed the passage of the active constituents, particularly polyphenols, through cutaneous tissue.

### Example 11

### Dispersion of Elicited, Comminuted Vine Cells in a Cosmetic Base

Vine cells were obtained as described in Examples 1A to 1I. The cells of these examples were used separately or in admixture for the preparation of a cosmetic composition. The cells were dispersed, after freeze-drying and comminution, in the following base:

| | |
|---|---|
| water | 46.89% |
| sodium lauryl sulphate (25%) | 36.40% |
| PEG-7 glyceryl cocoate | 2.00% |
| laureth-2 | 1.50% |
| laureth-11 sodium carboxylate | 4.00% |
| cocamidopropyl betaine & benzoic acid | 3.48% |
| sodium chloride | 1.60% |
| propylene glycol | 1.00% |
| perfume | 0.13% |
| PEG-40 hydrogenated castor oil | 0.50% |
| & propylene glycol & water | |
| oleth-10 | 0.50% |
| sodium phosphate | 0.30% |
| disodium phosphate | 0.08% |
| citric acid (50%) | 0.52% |
| sodium benzoate | 0.50% |
| vine cells | 0.20% |
| salicylic acid | 0.20% |
| phenoxyethanol | 0.20% |
| TOTAL | 100.00% |

The composition obtained exhibited a homogeneous dispersion of cells in the cream and a very fine particle size. A test for cleanliness showed the absence of germs and fungi as well as a remarkable stability of the composition. The result obtained from a transcutaneous investigation showed the passage of the active constituents, particularly polyphenols, through cutaneous tissue.

### Example 12

### Creams

aqueous phase A: demineralised water combined with a moisturising product
oleaginous phase B: emulsifier+emollient+oil
phase C: preservative, perfume
phase D: active substance: comminuted product of dedifferentiated, elicited vine cells, as a viscous suspension or a gel or a substantially dry powder.

### Example 13

### Lotions

Containing an aqueous phase A only: demineralised water, propylene glycol, preservative, perfume and active substance: comminuted product of dedifferentiated, elicited vine cells, as a viscous suspension or a gel or a substantially dry powder.

### Example 14

### Shampoos

Containing an aqueous phase A only, based on demineralised water, detergents, foaming agents, thickeners, perfume and active substance: comminuted product of dedifferentiated, elicited vine cells, as a viscous suspension or a gel or a substantially dry powder.

### Example 15

### Gels

Hydrogels and oleogels, obtained by the addition of emulsifiers and thickeners to the aqueous phase A or to the oleaginous phase B
phase C: perfume, preservative
phase D: comminuted product of dedifferentiated, elicited vine cells, as a viscous suspension or a gel or a substantially dry powder.

### Example 16

### Solutions

Solutions containing an aqueous phase A only, essentially based on demineralised water, perfume, preservative and active substance: comminuted, dedifferentiated, elicited vine cells, as a viscous suspension or a gel or a substantially dry powder.

### Example 17

### Milks

aqueous phase A: essentially based on deionised water
oleaginous phase B: oil+emulsifier+emollient
phase C: preservative+moisturising product
phase D: active substance: comminuted product of dedifferentiated, elicited vine cells, as a viscous suspension or a gel or a substantially dry powder.

In the examples given above relating to creams, gels or milks, the different phases A, B, C and D, in proportions which can vary according to the desired application, are mixed in a customary manner, as is usually effected by one skilled in this field.

With regard to lotions, solutions and shampoos, the composition for topical use contains different constituents, the content of which can be varied according to the application and which are mixed in the sole aqueous phase, as is usually effected by one skilled in this field.

The proportion of comminuted product of dedifferentiated, elicited vine cells, as a viscous suspension or a gel or a substantially dry powder, depends on the nature of the composition for topical use and on the desired application It advantageously ranges between 0.01 and 5%, but can amount to 25%.

The invention is obviously not limited to the examples given above, and it is possible to produce the composition for topical use in other forms, such as oils, ointments, lacquers, colours (foundation, powder, lipstick, pencil, mascara, eye shadow), which also fall within the scope of the invention.

Moreover, the invention is not limited to vine cells and can be applied to other types of plant cells provided that they can be obtained in dedifferentiated form and are capable of undergoing elicitation resulting in an accumulation of secondary metabolites in an amount sufficient quantity to facilitate biological activity in topical use.

In all cases, a composition for topical use is obtained which contains a comminuted product of dedifferentiated plant cells which are elicited, as a viscous suspension or as a gel or as a substantially dry powder.

### Example 18

Example 1 was repeated using dedifferentiated plant cells originating from different plant species or mixtures of different plant species. In these examples, peel, seed, beans, roots, leaves, stems, buds, fruits, skin or cuticle were used in order to obtain dedifferentiated plant cells.

The following Table lists the plant species used:

| Example 18 | Plant species |
|---|---|
| A | Rosmarinus |
| B | Coffea |
| C | Cacao |
| D | Mungo |
| E | Colchicum |
| F | Jasminuna + Iris |
| G | Capsicum |
| H | Pilocarpus |
| I | Sequoia |
| J | Solanum |
| K | Chlorophytum |
| L | Gingko |
| M | digitalis |
| N | Salvia |
| O | Taxus |
| P | Papaver |
| Q | Salvia + rosmarinus |
| R | Roses |
| S | Tea |
| T | Betula |
| U | Grapevine + citrus + ginko |

### Example 19

### Antioxidant Activity of Comminuted Products of Different Plant Species

The anti-radical activity of comminuted products derived from different plant species was studied in vitro. A SKINETHIC® reconstituted model epidermis was used to demonstrate this activity by the determination of malondialdehyde (MDA), after the induction thereof by ultraviolet B radiation. For each plant species, the epidermises were treated by a single concentration of 1% of comminuted product.

Induced Lipoperoxidation

The experimental conditions of Example 3 were repeated.

The results are given in the Table below:

| | Variation of |
|---|---|
| | MDA (in %) |
| | with respect to |
| | the negative |
| Plant species, 1% comminuted product | control |
| Negative control | — |
| Positive control + UVB (150 mJ/cm²) | 45% |
| | increase |
| 1% comminuted product of Cacao + UVB (150 mJ/cm²) | −29% |
| 1% comminuted product of Mungo + UVB | −22% |
| (150 mJ/cm²) | |
| 1% comminuted product of Colchicum + UVB | −26% |
| (150 mJ/cm²) | |
| 1% comminuted product of Jasminum + UVB | −17% |
| (150 mJ/cm²) | |
| 1% comminuted product of Capsicum + UVB | −43% |
| (150 mJ/cm²) | |
| 1% comminuted product of Pilocarpus + UVB | −41% |
| (150 mJ/cm²) | |
| 1% comminuted product of Sequoia + UVB | −59% |
| (150 mJ/cm²) | |
| 1% comminuted product of Solanum + UVB | −19% |
| (150 mJ/cm²) | |
| 1% comminuted product of Chlorophytum + UVB | −53% |
| (150 mJ/cm²) | |
| 1% comminuted product of Ginkgo + UVB | −52% |
| (150 mJ/cm²) | |
| 1% comminuted product of roses + UVB (150 mJ/cm²) | −25% |
| 1% comminuted product of Betula + UVB | −30% |
| (150 mJ/cm²) | |
| 1% comminuted product of digitalis + UVB | −33% |
| (150 mJ/cm²) | |
| 1% comminuted product of Salvia + UVB (150 mJ/cm²) | −47% |
| 1% comminuted product of Taxus + UVB (150 mJ/cm²) | −56% |
| 1% comminuted product of Papaver + UVB | −53% |
| (150 mJ/cm²) | |
| 1% comminuted product of Cannabis + UVB | −47% |
| (150 mJ/cm²) | |
| 1% comminuted product of Rosmarinus + UVB | −27% |
| (150 mJ/cm²) | |
| 1% comminuted product of Coffea + UVB | −34% |
| (150 mJ/cm²) | |
| 1% comminuted product of Arganier + UVB | −51% |
| (150 mJ/cm²) | |
| 1% comminuted product of Catharantus + UVB | −59% |
| (150 mJ/cm²) | |
| 1% comminuted product of Iris + UVB (150 mJ/cm²) | −19% |
| 1% comminuted product of Datura + UVB | −53% |
| (150 mJ/cm²) | |
| 1% comminuted product of Gloriosa + UVB | −12% |
| (150 mJ/cm²) | |
| 1% comminuted product of Medicago + UVB | −33% |
| (150 mJ/cm²) | |
| 1% comminuted product of Asparagus + UVB | −47% |
| (150 mJ/cm²) | |
| 1% comminuted product of Borago + UVB | −22% |
| (150 mJ/cm²) | |
| 1% comminuted product of Reseda + UVB | −13% |
| (150 mJ/cm²) | |
| 1% comminuted product of Amsonia + UVB | −26% |
| (150 mJ/cm²) | |
| 1% comminuted product of Erythrina + UVB | −21% |
| (150 mJ/cm²) | |
| 1% comminuted product of Coleus + UVB | −53% |
| (150 mJ/cm²) | |
| 1% comminuted product of Oneothera + UVB | −17% |
| (150 mJ/cm²) | |
| 1% comminuted product of Atropa + UVB | −23% |
| (150 mJ/cm²) | |
| 1% comminuted product of Theobroma + UVB | −17% |
| (150 mJ/cm²) | |
| 1% comminuted product of Wisteria + UVB | −43% |
| (150 mJ/cm²) | |
| 1% comminuted product of psoralea coryilfolia + UVB | −40% |
| (150 mJ/cm²) | |
| 1% comminuted product of vitex negundo + UVB | −42% |
| (150 mJ/cm²) | |
| 1% comminuted product of commiphora | −51% |
| wighii + UVB (150 mJ/cm²) | |
| 1% comminuted product of vanilla planifolia + UVB | −14% |
| (150 mJ/cm²) | |
| 1% comminuted product of marrubium vulgare + UVB | −28% |
| (150 mJ/cm²) | |
| 1% comminuted product of pilocarpus | −41% |
| jaborandi + UVB (150 mJ/cm²) | |

The results obtained show that at concentrations of 1% the comminuted products provide significant protection from lipoperoxidation induced by UVB radiation (150 mJ/cm²).

Under the experimental conditions employed, the comminuted products appeared to exhibit significant anti-radical activity in SKINETHIC® reconstituted epidermises after 24 hours of contact. This activity was detected by MDA determination.

In fact, the MDA determinations show that at concentrations of 1% the comminuted products tested significantly protect the cells from lipoperoxidation induced by ultraviolet B radiation (UVB 150 mJ/cm²) by decreasing the levels of induced MDA.

In conclusion, the comminuted products of the different plant species investigated exhibit an anti-radical effect under conditions of induction by ultraviolet B radiation. It is clear from this test that the comminuted products exhibit a significant anti-radical effect. Taking into account the model used (reconstituted epidermis), consideration can already be given to the use of these comminuted products in preparations for topical use at a minimum active dose of 0.1%.

### Example 20

Induced Lipoperoxidation

The experimental conditions of Example 3 were repeated, using combinations of aromatic plants with other species.

The results are given in the Table below:

| | Variation of |
|---|---|
| | MDA (in %) |
| | with respect to |
| | the negative |
| Plant species, 1% comminuted product | control |
| Negative control | — |
| Positive control [UVB (150 mJ/cm²)] | 45% |
| | increase |
| 1% comminuted product of Jasminum | −12% |
| sanbac + Ginko bilboa + UVB (150 mJ/cm²) | |
| 1% comminuted product of eucalyptus | −21% |
| punctata + psoralea coryfolia + UVB (150 mJ/cm²) | |
| 1% comminuted product of lavandula | −17% |
| angustifolia + Vitex negundo + UVB (150 mJ/cm²) | |
| 1% comminuted product of citrus limon + | −24% |
| sequoia + UVB (150 mJ/cm²) | |

### Example 21

Examples 1A to 1I were repeated, except that the dedifferentiated, elicited and washed cells were dried in an atmosphere (of nitrogen, for example) at a temperature of about 30° C., in order to reduce the water content of the cells to 5% by weight, 10% by weight and 15% by weight, respectively. The membrane structure of the cell was thus preserved.

The dedifferentiated, elicited, washed and dried cells were subsequently subjected to comminution.

### Example 22

Example 20 was repeated, except that the dedifferentiated, elicited, washed and dried cells (with their membrane structure preserved) were mixed with one or more excipients and/or compounds of a cosmetic composition before the comminution stage.

The following list gives some of the excipients mixed with the cells before the latter were comminuted.
sodium lauryl sulphate (25% aqueous solution, for example)
PEG-7 glyceryl cocoate
antioxidant (containing an aqueous solution of vitamin E)
laureth-2
sodium laureth-11 carboxylate
cocamidopropyl betaine
betaine (glycinobetaine)
essential oil
propylene glycol
ethanol
PEG40 hydrogenated castor oil
vegetable oil (coconut oil, olive oil, etc.)
essential oil
mixtures of one or more of the compounds cited above with each other and/or with water.

The amount of excipients and/or of water added to the cells can vary, for example, between 10% of the weight of the dried cells to be comminuted and 100% of said weight, or even more.

The use of one or more surface active agents with one or more antioxidants appears to be of interest in order to facilitate comminution of the cells, particularly the membrane, to facilitate the release of phytoalexins attached or linked to a membrane, and to reduce or to avoid any problem of degradation of the compounds due to humidity. It is subsequently possible to extract phytoalexins from the comminuted product by an extraction stage (for example successive ethanol extraction stages and filtration).

This example is therefore an example of a method of obtaining phytoalexin(s), in which:
dedifferentiated plant cells are placed in a culture medium,
after and/or during culture, the dedifferentiated cells are elicited in the culture medium,
the dedifferentiated, elicited cells are separated from the culture medium,
the dedifferentiated, elicited cells are optionally subjected to one or more washing stages,
the dedifferentiated, elicited cells are advantageously dried, preferably freeze-dried,
the dedifferentiated, elicited cells are comminuted in order to form a comminuted product, and
the comminuted product is subjected to an extraction stage in order to extract one or more phytoalexins from the comminuted product, possibly after a stage of introducing the comminuted cells into a medium, particularly an aqueous and/or alcoholic medium.

### Example 23

Examples 1A to 1I were repeated, except that the dedifferentiated, elicited cells were washed and dried (by means of a current of nitrogen at 30° C.) to eliminate the wash water present on the outside of the cell membranes and to reduce the water content present in the cells, of 0%, 10%, 25%, 50% and 75%, respectively. The cells were subsequently comminuted. The comminuted product thus obtained contained substantially all the components present in the cells, either with a reduced water content or with a water content corresponding to the water present in normal cells (normal cell water content).

### Example 24

Examples 1A to 1I and 18 have been repeated for the preparation of elicited plant cells. The elicited cells were not submitted to a drying, nor to a comminution. The culture containing the elicited cells was filtered so as to recover the cells. The cells have been washed and mixed thereafter with propyleneglycol. A suspension containing about 30% elicited cells was so obtained.

The suspension containing the elicited cells was then submitted to an electroporation step with an AC current of 50V.

The so obtained composition (propylene glycol+perforated cells) can thereafter be used as such, or as intermediate material for the preparation of skin compositions similar to the compositions of examples 8 to 17.

### Example 25

Example 24 has been repeated, except that the recovered and washed cells were submitted to an electroporation (DC current 50V, pulse of 10 μs).

Said electroporated cells have then been used for the preparation of skin compositions similar to the compositions of examples 8 to 17, the comminuted cells of said example being only replaced by the electropored cells.

Examples 24 and 25 have been repeated by using other plant species, namely the plant species listed in Example 18.

1. A composition for topical application containing at least one comminuted product of elicited dedifferentiated plant cells, whereby said dedifferentiated plant cells are elicited in vitro in a culture in order to synthesise at least one phytoalexin, whereby the communited product of elicited dedifferentiated plant cells are at least in a form suitable for being dispersed in said composition.

2. The composition of claim 1, in which said communited product of elicited dedifferentiated plant cells comprises the at least one phytoalexin synthesised in vitro and comprises at least 95% by weight of the entirety of the dry materials derived from the elicited dedifferentiated comminuted plant cells.

3. The composition of claim 1, in which said communited product of elicited dedifferentiated plant cells comprises the at least one phytoalexin synthesised in vitro and comprises at least 97% by weight of the entirety of the dry materials derived from the elicited dedifferentiated comminuted plant cells.

4. The composition of claim 1, in which said communited product of elicited dedifferentiated plant cells comprises the at least one phytoalexin synthesised in vitro and comprises at least 99% by weight of the entirety of the dry materials derived from the elicited dedifferentiated comminuted plant cells.

5. The composition of claim 1, in which the communited product of elicited dedifferentiated plant cells are dispersed in said composition.

6. The composition of claim 1, in which the dedifferentiated plant cells are elicited in vitro in a culture in order to synthesise at least one phytoalexin, after being effected after an in vitro stage of plant cell culture without elicitation.

7. The composition of claim 1, which is a cosmetic composition.

8. The composition of claim 1, in which that the communited product of elicited dedifferentiated plant cells comprises particles selected from the group consisting of particles derived from vacuoles, particles derived from cytoplasm, particles derived from pecto-cellulose membrane and mixtures thereof, whereby said comminuted product contains at least 0.1% by weight of phytoalexin.

9. The composition of claim 1, in which that the communited product of elicited dedifferentiated plant cells comprises particles selected from the group consisting of particles derived from vacuoles, particles derived from cytoplasm, particles derived from pecto-cellulose membrane and mixtures thereof, whereby said comminuted product contains at least 0.1% by weight of phytoalexins.

10. The composition of claim 1, in which the comminuted product is a comminuted product of dedifferentiated cells which are elicited in vitro, said cells being at least partially dried.

11. The composition of claim 10, in which the comminuted product is a comminuted product of dedifferentiated cells which are elicited in vitro, said cells being substantially completely dried before being comminuted.

12. The composition of claim 1, in which the comminuted product is a comminuted product of dedifferentiated cells which are elicited in vitro, said cells being freeze-dried before being comminuted.

13. The composition of claim 1, which contains 0.005 to 25% by weight of a comminuted product of dedifferentiated plant cells which are elicited in vitro, said weight being calculated in dry form.

14. The composition of claim 1, which contains 0.005 to 5% by weight of a comminuted product of dedifferentiated plant cells which are elicited in vitro, said weight being calculated in dry form.

15. The composition of claim 1, which contains a substantially dry comminuted product of elicited dedifferentiated plant cells which are elicited in vitro, said substantially dry comminuted product having a water content less than 25% by weight.

16. The composition of claim 1, which contains a substantially dry comminuted product of elicited dedifferentiated plant cells which are elicited in vitro, said substantially dry comminuted product having a water content less than 15% by weight.

17. The composition of claim 1, which contains a substantially dry comminuted product of elicited dedifferentiated plant cells which are elicited in vitro, said substantially dry comminuted product having a water content less than 10% by weight.

18. The composition of claim 1, in which the communited product has an average particle size of less than 100 μm.

19. The composition of claim 1, in which the communited product has an average particle size of less than 10 μm.

20. The composition of claim 1, in which the communited product has an average particle size of less than 1 μm.

21. The composition of claim 1, in which the communited product has a particle size distribution such that 90% by weight of the particles have a particle size ranging from the average particle size −25% to the average particle size +25%.

22. The composition of claim 1, in which said comminuted product of dedifferentiated plant cells which are elicited in vitro contains at least one phytoalexin synthesised by the in vitro elicitation of dedifferentiated plant cells.

23. The composition of claim 1, in which said comminuted product of dedifferentiated, elicited plant cells is a comminuted product of dedifferentiated plant cells which are elicited in vitro by an agent in the culture medium, said comminuted product being substantially free from said agent after elicitation.

24. The composition of claim 23, in which the dedifferentiated cells are elicited in vitro by a volatile agent.

25. The composition of claim 1, in which said comminuted product of dedifferentiated plant cells which are elicited in vitro contains at least one compound selected from the group consisting of terpenic compounds, tannic compounds, polyphenolic compounds and mixtures thereof, said compound being synthesised by the in vitro elicitation of the dedifferentiated plant cells in their culture medium.

26. The composition of claim 1, in which the comminuted product of dedifferentiated plant cells which are elicited in vitro is in a form selected from the group consisting of viscous suspensions, gels and substantially dry powders.

27. The composition of claim 1, in which the comminuted product comprises at least a comminuted product of dedifferentiated vine cells which are elicited in vitro.

28. The composition of claim 1, which contains a comminuted product of dedifferentiated cells which are cultivated and elicited in their in vitro culture medium.

29. The composition of claim 28, in which said comminuted product is substantially free from culture medium.

30. The composition of claim 1, which contains a comminuted product of dedifferentiated cells which are elicited in vitro, said comminuted product containing at least 0.1% by weight of stilbenes based on the dry weight of the comminuted, dedifferentiated, elicited cells.

31. The composition of claim 1, which contains a comminuted product of dedifferentiated cells which are elicited in vitro, said comminuted product containing at least 0.2% by weight of stilbenes based on the dry weight of the comminuted, dedifferentiated, elicited cells.

32. The composition of claim 1, which contains a comminuted product of dedifferentiated cells which are elicited in vitro, said comminuted product containing at least 0.5% by weight of stilbenes based on the dry weight of the comminuted, dedifferentiated, elicited cells.

33. The composition of claim 1, in which the comminuted product of dedifferentiated, elicited cells is derived from the culture of dedifferentiated plant cells, which are elicited and then dried, of at least one species selected from the group consisting of Salvia, Coleus, Rosmarinus, Gingko, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, betula, tea and mixtures of cells of such species.

34. The composition of claim 1, which comprises at least one excipient.

35. A method of preparing a composition for topical use containing at least one excipient and at least one comminuted product of elicited dedifferentiated plant cells, whereby said dedifferentiated plant cells are elicited in vitro in a culture in order to synthesise at least one phytoalexin, whereby the communited product of elicited dedifferentiated plant cells are at least in a form suitable for being dispersed in said composition, in which:
dedifferentiated plant cells are placed and growth in an in vitro culture medium,
the dedifferentiated plant cells are elicited in their culture medium during a period of time sufficient for the synthesis of elicited dedifferentiated plant cells with a sufficient quantity of metabolites, and
the dedifferentiated plant cells elicited in the in vitro culture medium are mixed with one or more excipients in order to prepare a cosmetic composition
whereby the elicited dedifferentiated plant cells are comminuted in at least one communiting step selected from the group consisting of communiting step before mixing the elicited dedifferentiated plant cells with at least one excipient, communiting step after mixing the elicited dedifferentiated plant cells with at least one excipient, communiting step after a drying stage, and combinations thereof.

36. The method of claim 35, in which said plant cells which are elicited in vitro are subjected to a drying step, followed by comminution.

37. The method of claim 35, in which the plant cells are elicited in their in vitro culture medium by means of an agent which, after extracting the elicited cells from the culture medium whilst retaining the membrane structure of the cells, does not occur in the elicited cells.

38. The method of claim 35, in which the dedifferentiated plant cells are placed in a growth culture in vitro, are elicited in said in vitro culture medium, are dried, are then comminuted and dispersed in a composition for the treatment of the human body.

39. The method of claim 38, in which the elicited dedifferentiated plant cells are at least submitted to one washing step before being comminuted.

40. The method of claim 38, in which the cells are dried by freeze-drying before being communited.

41. The method of claim 35, in which the dedifferentiated plant cells are elicited in their culture medium by an eliciting agent which does not form an impurity in the comminuted product of dedifferentiated and elicited cells.

42. The method of claim 35, in which the dedifferentiated plant cells are elicited in their culture medium by an eliciting means which does not form an impurity in the comminuted product of dedifferentiated and elicited cells.

43. A method of obtaining phytoalexin(s), comprising at least the following steps:
dedifferentiated plant cells are placed in an in vitro culture medium,
the dedifferentiated cells are elicited in the culture medium,
the dedifferentiated, elicited cells are separated at least partly from the culture medium,
the dedifferentiated, elicited cells are comminuted in order to form a comminuted product, and
the comminuted product is subjected to at least one extraction to extract at least one phytoalexin from the comminuted product.

44. The method of claim 43, in which the comminuted product is subjected to an extraction after a stage of introducing the comminuted cells into a medium.

45. The method of claim 43, in which the comminuted product is subjected to an extraction after a stage of introducing the comminuted cells into a medium selected from the group consisting of aqueous mediums, alcoholic mediums and mixtures thereof.

46. The method of claim 43, in which the dedifferentiated, elicited cells separated at least partly from the culture medium are submitted to at least one washing step, before being comminuted.

47. The method of claim 43, in which the dedifferentiated, elicited cells separated at least partly from the culture medium are submitted to at least one drying step, before being comminuted.

48. The method of claim 43, in which the dedifferentiated, elicited cells separated at least partly from the culture medium are submitted to at least one washing step followed by a drying step, before being comminuted.

49. The method of claim 43, in which the dedifferentiated, elicited cells separated at least partly from the culture medium are submitted to at least one freeze-drying step, before being comminuted.

50. The method of claim 43, in which the dedifferentiated plant cells are elicited in their culture medium by an eliciting agent which does not form an impurity in the comminuted product of dedifferentiated and elicited cells.

51. The method of claim 43, in which the stage of eliciting said dedifferentiated plant cells in their in vitro culture medium is controlled in order to obtain a medium containing at least 0.1% by weight of stilbenes based on the dry weight of the dedifferentiated, elicited plant cells.

52. The method of claim 43, in which the stage of eliciting said dedifferentiated plant cells in their in vitro culture medium is controlled in order to obtain a medium containing at least 0.2% by weight of stilbenes based on the dry weight of the dedifferentiated, elicited plant cells.

53. The method of claim 43, in which the stage of eliciting said dedifferentiated plant cells in their in vitro culture medium is controlled in order to obtain a medium containing at least 0.5% by weight of stilbenes based on the dry weight of the dedifferentiated, elicited plant cells.

54. The method of claim 43, in which the comminuted product is derived from the culture of dedifferentiated plant cells, which are elicited and then dried, of at least one species selected from the group consisting of Salvia, Coleus, Rosmarinus, Gingko, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, cominiphora wighii, eucalyptus punctata, lavandula angustifolia, citrus limon, vanilla planifolia, marrubium vulgare, pilocarpus jaborandi, roses, betula, tea, and mixtures thereof.

55. A comminuted product of dedifferentiated plant cells which are elicited in an in vitro culture medium and then dried, wherein said comminuted product containing at least one phytoalexin comprises at least 95% by weight of the entirety of the dry materials derived from the dedifferentiated plant cells which are elicited in vitro, said comminuted product being in a form suitable for being dispersed in a composition selected from the group consisting of cosmetic compositions and pharmaceutical compositions.

56. The product of claim 55, which comprises at least 97% by weight of the entirety of the dry materials derived from the dedifferentiated plant cells which are elicited in vitro.

57. The product of claim 55, which comprises at least 99% by weight of the entirety of the dry materials derived from the dedifferentiated plant cells which are elicited in vitro.

58. The product of claim 57, which is free from elicitation agent.

59. The product of claim 57, which is free from culture medium.

60. The product of claim 57, which consists of particles with an average particle size of less than 10 μm.

61. The product of claim 57, which contains at least 0.1% by weight of stilbenes based on the dry weight of the cells.

62. The product of claim 57, which contains at least 0.2% by weight of stilbenes based on the dry weight of the cells.

63. The product of claim 57, which contains at least 0.5% by weight of stilbenes based on the dry weight of the cells.

64. A composition for topical application containing at least one comminuted product of in vitro elicited dedifferentiated plant cells containing at least one phytoalexin, whereby said comminuted product contains at least 95% by weight of the entirety of the dry materials derived from comminuted, dedifferentiated plant cells which are elicited in vitro, and whereby said comminuted product contains at least 0.1% by weight of stilbenes based on the dry weight of the cells.

65. The composition of claim 64, in which said comminuted product contains at least 0.2% by weight of stilbenes based on the dry weight of the cells.

66. The composition of claim 64, in which said comminuted product contains at least 0.5% by weight of stilbenes based on the dry weight of the cells.

67. The composition of claim 64, in which said comminuted product contains at least 97% by weight of the entirety of the dry materials derived from comminuted dedifferentiated plant cells which are elicited in vitro.

68. The composition of claim 64, in which said comminuted product contains at least 99% by weight of the entirety of the dry materials derived from comminuted dedifferentiated plant cells which are elicited in vitro.

69. The composition of claim 64, in which said comminuted product is a comminuted product of dedifferentiated plant cells, which are elicited and at least dried before being communited, and in which the dedifferentiated plant cells are selected from the group of species consisting of Salvia, Coleus, Rosmarinus, Ginkgo, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, betula, tea, and mixtures thereof.

70. The composition of claim 64, which is a cosmetic composition.

71. A composition for topical application containing at least elicited dedifferentiated plant cells, whereby said dedifferentiated plant cells are elicited in vitro in a culture in order to synthesise at least one phytoalexin, whereby the elicited dedifferentiated plant cells are permeabilized and at least in a form suitable for being dispersed in said composition.

72. The composition of claim 71, in which the permeabilized elicited dedifferentiated plant cells are dispersed in said composition.

73. The composition of claim 71, in which the dedifferentiated plant cells are elicited in vitro in a culture in order to synthesise at least one phytoalexin, after an in vitro stage of plant cell culture without elicitation.

74. The composition of claim 71, which is a cosmetic composition.

75. The composition of claim 71, in which the permeabilized elicited dedifferentiated plant cells are electro pored cells.

76. The composition of claim 71, in which the permeabilized elicited dedifferentiated plant cells are at least partially dried.

77. The composition of claim 71, in which the dedifferentiated cells which are elicited in vitro are substantially completely dried before permeabilized.

78. The composition of claim 71, which contains 0.005 to 25% by weight of a permeabilized elicited dedifferentiated plant cells, said weight being calculated in dry form.

79. The composition of claim 71, which contains 0.005 to 5% by weight of a permeabilized elicited dedifferentiated plant cells, said weight being calculated in dry form.

80. The composition of claim 71, which contains a substantially dry permeabilized elicited dedifferentiated plant cells which are elicited in vitro, said substantially dry plant cells having a water content less than 25% by weight.

81. The composition of claim 71, which contains a substantially dry permeabilized elicited dedifferentiated plant cells which are elicited in vitro, said substantially dry plant cells having a water content less than 15% by weight.

82. The composition of claim 71, which contains a substantially dry permeabilized elicited dedifferentiated plant cells which are elicited in vitro, said substantially dry plant cells having a water content less than 10% by weight.

83. The composition of claim 71, in which said permeabilized dedifferentiated plant cells which are elicited in vitro contains at least one phytoalexin synthesised by the in vitro elicitation of dedifferentiated plant cells.

84. The composition of claim 71, in which said permeabilized elicited dedifferentiated plant cells is dedifferentiated plant cells which are elicited in vitro by an agent in the culture medium before being permeabilized, whereby said dedifferentiated plant cells are substantially free from said agent after elicitation.

85. The composition of claim 84, in which the dedifferentiated cells are elicited in vitro by a volatile agent.

86. The composition of claim 71, in which said dedifferentiated plant cells which are elicited in vitro contains at least one compound selected from the group consisting of terpenic compounds, tannic compounds, polyphenolic compounds and mixtures thereof, said compound being synthesised by the in vitro elicitation of the dedifferentiated plant cells in their culture medium.

87. The composition of claim 71, in which the dedifferentiated plant cells which are elicited in vitro and permeabilized are in a form selected from the group consisting of viscous suspensions, gels and substantially dry powders.

88. The composition of claim 71, which comprises dedifferentiated vine cells which are elicited in vitro and permeabilized.

89. The composition of claim 71, which contains dedifferentiated plant cells which are cultivated and elicited in their in vitro culture medium, and submitted to a permeabilizing step.

90. The composition of claim 89, which is substantially free from culture medium.

91. The composition of claim 71, which comprises permeabilized elicited dedifferentiated cells which are elicited in vitro containing at least 0.1% by weight of stilbenes based on the dry weight of the dedifferentiated, elicited cells.

92. The composition of claim 71, which comprises permeabilized elicited dedifferentiated cells which are elicited in vitro containing at least 0.2% by weight of stilbenes based on the dry weight of the dedifferentiated, elicited cells.

93. The composition of claim 71, which comprises permeabilized elicited dedifferentiated cells which are elicited in vitro containing at least 0.5% by weight of stilbenes based on the dry weight of the dedifferentiated, elicited cells.

94. The composition of claim 1, in which the permeabilized dedifferentiated, elicited plant cells is derived from the culture of dedifferentiated plant cells, which are elicited and permeabilized, of at least one species selected from the group consisting of Salvia, Coleus, Rosmarinus, Gingko, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, betula, tea and mixtures of cells of such species.

95. The composition of claim 71, which comprises at least one excipient.

96. The composition of claim 71, which comprises at least a glycol ester.

97. The composition of claim 71, which comprises at least propylene glycol.

98. The composition of claim 71, in which the permeabilized elicited dedifferentiated plant cells are provided with a coating.

99. A method of preparing a composition for topical use containing at least one excipient and at least one permeabilized elicited dedifferentiated plant cells, whereby said dedifferentiated plant cells are elicited in vitro in a culture in order to synthesise at least one phytoalexin, whereby the permeabilized elicited dedifferentiated plant cells are at least in a form suitable for being dispersed in said composition, in which:
dedifferentiated plant cells are placed and growth in an in vitro culture medium,
the dedifferentiated plant cells are elicited in their culture medium during a period of time sufficient for the synthesis of elicited dedifferentiated plant cells with a sufficient quantity of metabolites, and
the dedifferentiated plant cells elicited in the in vitro culture medium are mixed with one or more excipients in order to prepare a cosmetic composition
whereby the elicited dedifferentiated plant cells are permeabilized in at least one permeabilizing step selected from the group consisting of permeabilizing step before mixing the elicited dedifferentiated plant cells with at least one excipient, permeabilizing step after mixing the elicited dedifferentiated plant cells with at least one excipient, permeabilizing step after a drying stage, and combinations thereof.

100. The method of claim 99, in which said plant cells which are elicited in vitro are subjected to a filtration step, before being permeabilized.

101. The method of claim 99, in which the plant cells are elicited in their in vitro culture medium by means of an agent which, after extracting the elicited cells from the culture medium whilst retaining the membrane structure of the cells, does not occur in the elicited cells.

102. The method of claim 99, in which the dedifferentiated plant cells are placed in a growth culture in vitro, are elicited in said in vitro culture medium, are at least partly separated from the growth culture, are then permeabilized and dispersed in a composition for the treatment of the human body.

103. The method of claim 99, in which the elicited dedifferentiated plant cells are at least submitted to one washing step before being permeabilized.

104. The method of claim 103, in which the cells are at least partly dried before being permeabilized.

105. The method of claim 99, in which the dedifferentiated plant cells are elicited in their culture medium by an eliciting agent which does not form an impurity in the permeabilized dedifferentiated and elicited cells.

106. The method of claim 99, in which the dedifferentiated plant cells are elicited in their culture medium by an eliciting means which does not form an impurity for the composition.

107. The method of claim 99, in which the dedifferentiated plant cells are at least one species selected from the group consisting of Salvia, Coleus, Rosmarinus, Gingko, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, cominiphora wighii, eucalyptus punctata, lavandula angustifolia, citrus limon, vanilla planifolia, marrubium vulgare, pilocarpus jaborandi, roses, betula, tea, and mixtures thereof.

108. A permeabilized product of dedifferentiated plant cells which are elicited in an in vitro culture medium and thereafter permeabilized, wherein said permeabilized product containing at least one phytoalexin, said permeabilized product being in a form suitable for being dispersed in a composition selected from the group consisting of cosmetic compositions and pharmaceutical compositions.

109. The product of claim 108, which is free from elicitation agent.

110. The product of claim 108, which is free from culture medium.

111. The product of claim 108, which contains at least 0.1% by weight of stilbenes based on the dry weight of the cells.

112. The product of claim 108, which contains at least 0.2% by weight of stilbenes based on the dry weight of the cells.

113. The product of claim 108, which contains at least 0.5% by weight of stilbenes based on the dry weight of the cells.

114. The product of claim 108, which comprises a glycol ester.

115. The product of claim 108, which comprises propyleneglycol.

116. The product of claim 108, which comprises from 5 to 30% by weight of permeabilized dedifferentiated plant cells elicited in vitro.

117. The product of claim 108, in which the dedifferentiated plant cells are selected from the group of species consisting of Salvia, Coleus, Rosmarinus, Ginkgo, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, betula, tea, and mixtures thereof.

118. A composition for topical application containing at least one permeabilized product of in vitro elicited dedifferentiated plant cells containing at least one phytoalexin, whereby said permeabilized product contains at least 0.1% by weight of stilbenes based on the dry weight of the cells.

119. The composition of claim 118, in which said permeabilized product contains at least 0.2% by weight of stilbenes based on the dry weight of the cells.

120. The composition of claim 118, in which said permeabilized product contains at least 0.5% by weight of stilbenes based on the dry weight of the cells.

121. The composition of claim 118, in which said permeabilized product is a permeabilized product of dedifferentiated plant cells, which are elicited in vitro, and in which the dedifferentiated plant cells are selected from the group of species consisting of Salvia, Coleus, Rosmarinus, Ginkgo, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, betula, tea, and mixtures thereof.

122. The composition of claim 118, which is a cosmetic composition.
